(19) United States
(12) Patent Application Publication
(10) Pub. No.: US 2026/0315764 A1
(43) Pub. Date: Oct. 15, 2026

(54) SYSTEM AND METHOD FOR SUSTAINED RELEASE OF AGENTS IN THE BLADDER

(71) Applicant: WATERSHED MEDICAL, INC., Bellevue, WA (US)

(72) Inventors: Christopher R. Kinsella, JR., Bellevue, WA (US); Saniya Ali, North Bend, WA (US); Jackson Valenzuela, Seattle, WA (US); Adriana Yarkin, II, Vashon, WA (US); Clay Justin Nolan, Stony Brook, NY (US)

(73) Assignee: WATERSHED MEDICAL, INC., Bellevue, WA (US)

(21) Appl. No.: 19/643,747

(22) Filed: Apr. 9, 2026

Related U.S. Application Data
(60) Provisional application No. 63/786,253, filed on Apr. 9, 2025.

(51) Int. Cl. A61M 31/00; A61L 31/10; A61L 31/16

(52) U.S. Cl. CPC A61M 31/002; A61L 31/10; A61L 31/16; A61L 2420/02; A61L 2420/06; A61M 2205/0238; A61M 2210/1085

(57) ABSTRACT

Local delivery of therapeutics into the urinary bladder can increase tissue concentration while minimizing systemic exposure. However, bladder implants must be securely retained within the bladder for the duration of therapy while ensuring controlled passive expulsion without requiring medical intervention. Conventional devices have limitations such as premature disassembly, unpredictable expulsion timing and inconsistent performance due to swelling variations. The drug products described herein overcome these limitations. They can be administered through the urethra in a compact form to treat diseases of the bladder, kidneys and urinary tract. Within the bladder, the products conform to a larger size so that they are retained while a drug is released. After a treatment period, individual components of the particles separate from one another and are voided in the urine.

### RELATED APPLICATIONS

The application claims priority to U.S. Provisional Patent Application No. 63/786,253 filed Apr. 9, 2025, the contents of which are herein incorporated by reference.

### FIELD OF THE INVENTION

The invention relates generally to medical devices, and, more particularly, to devices and methods for intravesical administration of an active agent to treat or prevent a disorder of the urinary system. The invention further relates to modular, geometry-programmed, degradable, or disassemble-able intravesical drug-delivery systems configured to provide sustained release, staged release, programmable detachment, and safe elimination.

### BACKGROUND

A urinary tract infection (“UTI”) is an infection that affects part of the urinary tract. When it affects the lower urinary tract it is known as a bladder infection (i.e., cystitis) and when it affects the upper urinary tract it is known as a kidney infection (i.e., pyelonephritis). Symptoms from a lower urinary tract infection include pain with urination, frequent urination, and feeling the need to urinate despite having an empty bladder. The most common cause of UTI is Escherichia coli, though other bacteria or fungi can also be the cause. Risk factors include female anatomy, sexual intercourse, diabetes, obesity, and family history. In uncomplicated cases, UTIs are treated with a short course of antibiotics (e.g., nitrofurantoin). Nearly one in five adults develops a UTI, and many of these individuals are at risk for a recurring infection.

The prevalence of UTI risk increases with age. They are particularly common in older adults who use catheters or live in a nursing home or other full-time care facilities. Other conditions common in older adults (e.g., Alzheimer's disease, Parkinson's disease and diabetes) can lead to urinary retention or neurogenic bladder which increases the risk of UTIs. More than 10 percent of women over age 65 report having a UTI within the past year. That number increases to almost 30 percent in women over 85. Men also tend to experience more UTIs as they age. According to a recent study, more than one-third of all infections in people in nursing homes are UTIs. In many cases, chronic urinary tract infections can require continuous medication, which leads to progressive levels of resistance to antibiotics and ultimately to kidney problems.

If not treated timely or properly, an infection from a UTI can spread. A UTI can ultimately lead to a bladder infection. This presents the risk of the infection spreading to the kidney via the ureter which can lead to more serious consequences including kidney failure and sepsis (i.e., urosepsis). This occurs frequently in elderly patients. In many cases, elderly patients do not present typical signs of infection until they've become septic at which point hospitalization is needed.

UTIs and complications caused by UTIs are also expensive to treat as they often require hospitalization. In many cases, a UTI is only treated after the infection has developed and becomes symptomatic. Current treatments aimed at preventing the formation of UTIs require continuous prophylactic oral medication that have side-effects common with long-term medication. Further, long term consumption of antibiotics can lead to drug resistant bacteria. In many cases, UTIs reduce the quality of life, in particular when kidney damage leads to kidney failure requiring dialysis or implantation of a donor kidney.

Conventionally, UTIs are treated with oral antibiotics. A high dose of antibiotics is needed so that an effective amount reaches the urinary tract. With systemic delivery of drugs to treat UTIs, the volume of distribution of the therapy is equal to the total amount of urine that enters the bladder. Therefore, the therapy is voided with each emptying of the bladder and requires constant replenishment. In other cases, the drug target can be limited (i.e., the bladder wall) but cannot practically be reached without raising the concentration of the drug in the urine to the desired therapeutic concentration.

Intravesical therapy refers to a treatment wherein a therapeutic is put directly into the bladder (e.g., through a catheter or similar device) rather than being given orally or injected into a vein. Intravesical therapy is an attractive option for treating UTIs because it allows local delivery of a medicament while minimizing side effects. Intravesical administration of pharmaceutically active agents can also be beneficial for treating other diseases of the bladder/urinary tract such as interstitial cystitis, overactive bladder and cancers.

Efforts at treating UTIs via intravesical therapy have had limited success. Antibiotics injected into the bladder are generally voided before they can induce a significant therapeutic effect. Implantable devices require surgical procedures for their implantation and removal. Recent improvements include implantable devices that gradually dissolve or degrade as an active agent is released. For example, WO2022216287A1 describes a controlled release pharmaceutical formulation that is administered into the bladder of a patient. The formulation includes particles that are buoyant in urine and can remain in a patient's bladder for a course of antibiotic treatment. However, the particles are prone to being expelled prematurely during urination. Moreover, the retention time cannot be controlled.

Other bladder-retained drug-delivery devices (e.g., monolithic rods, rings, dog-bone devices) have also been developed. However, these devices present limitations including:
uncontrolled retention or premature expulsion,
sedimentation at the bladder floor,
single-kinetic drug release profiles,
risk of obstruction or irritation,
inability to control timing of clearance from the bladder, and
manufacturing challenges with drug uniformity.

Accordingly, there is also a need for small particles that are (a) relatively easy to administer into the bladder, (b) that remain in the bladder while an agent is released and (c) are thereafter expelled in the urine. The particles should also be versatile, allowing for their use with different pharmaceutical agents. Further, the particles should be capable of modification to control their retention time. The compositions and methods described herein satisfy these objectives. The system and methods described herein can also be used to treat various ailments of the bladder/urethra/kidneys such as, for example, infection, cancer, overactive bladder and cystitis.

### SUMMARY OF THE INVENTION

The inventions described and claimed herein have many attributes and embodiments including, but not limited to, those set forth or described or referenced in this brief summary. The inventions described and claimed herein are not limited to, or by, the features or embodiments identified in this summary, which is included for purposes of illustration only and not restriction.

There is a need for an intravesical system with drug products that is easily inserted, retained for controlled duration, release drugs programmably, disassemble safely, and are passively eliminated. The system should have predictable expulsion, modular safety with multimodal PK programming.

Embodiments of the invention include devices and methods for intravesical delivery of therapeutic agents into the bladder. A plurality of buoyant (or semi-buoyant) drug products can be temporarily implanted inside the bladder and float within fluid inside the bladder. The drug products can release a therapeutic agent or drug within the bladder by direct absorption, passive or active dissolution, mechanical fracture or other means.

In aspects, the drug products are passively released (i.e., without requiring a removal procedure) from the bladder after the retention period.

In aspects, particles can be attached to one another in a first configuration that is conducive to transurethral administration. After the particles enter the urinary bladder of the subject, they can expand to a second configuration that is larger and resistant to excretion. During a retention phase, an active agent can be released from the particles into the bladder. Thereafter, the particles can gradually separate from one another so that they are excreted during urination.

In aspects, the modular intravesical drug-delivery device includes (a) a plurality of rod-shaped modules, each with a drug-containing polymeric body and (b) one or more hinge elements that connect the adjacent modules. Each hinge element can be configured to enable unidirectional bending, resist bending in an opposite direction and/or undergo controlled weakening or degradation. The modules can be dimensioned such that the assembled device has a diameter large enough to prevent excretion from the bladder. The hinge elements can be further configured to permit detachment of the modules, which may be sequential, simultaneous, or a combination thereof, optionally according to a desired (i.e., predetermined) timing profile.

In aspects, the device is configured to assume three distinct geometric configurations: (i) an insertion configuration, in which the modules are arranged substantially collinearly for transurethral administration, having a maximum cross-sectional profile of less than about 10 mm; (ii) a retention configuration, in which at least two modules are oriented non-collinearly (e.g., substantially orthogonally) with respect to one another by the hinge elements, such that the minimum cross-sectional profile of the device in any orientation is at least about 16 mm so as to resist excretion through the urethra during voiding; and (iii) an elimination configuration, after detachment of the modules from one another, in which each individual module has a maximum cross-sectional profile of less than about 6 mm for passive excretion through the urethra.

Embodiments also include a hinge for an intravesical modular device. The hinge can include (a) an asymmetric geometric cross-section enabling bending in a first direction and resisting bending in a second direction and (b) a degradable polymer composition comprising poly(caprolactone-co-glycolide) (PCL-GA), polycaprolactone (PCL), glycolic acid (GA), or combinations thereof. The hinge can include a thickness gradient or density gradient such that degradation rate varies along its length, producing time-dependent weakening and disassembly.

Embodiments also include a multi-segment floating intravesical system that includes a plurality (e.g., two) of rod-shaped modules with a density about 0.80 g/cm³ and less than about 1.05 g/cm³ (e.g., between about 0.80 and about 0.85, between about 0.85 and about 0.90, between about 0.90 and about 0.95, between about 0.95 and about 1.00, or between about 1.00 and less than about 1.05 g/cm³). Each module is buoyant before and after detachment from one another and is independent of the degradation state or drug-release state of the module.

Embodiments also include a drug-delivery system that includes multiple (e.g., two) drug-loaded modules arranged in a chain. The drug loading across modules can be uniform distribution, tapered gradients, stratified patterns, or module-specific loading. Drug-release kinetics can be determined by, for example, module position in the chain, hinge detachment timing, coating thickness and polymer composition of each module.

Embodiments also include a coated intravesical chain device that includes modules coated with a polymer such as EVA, PCL-GA, polyurethane, PLGA, or blends thereof. The different modules within the chain can possess different coating thicknesses, porosities, or compositions, thereby enabling one or more of
a) burst/induction release from a first module,
b) sustained release from a second module,
c) delayed or pulsatile release from a third module, or
d) multi-drug sequential release.

Accordingly, embodiments include an intravesical drug delivery device (“4RC”), that includes: (a) a plurality of elongate modules, each module comprising a polymeric matrix and at least one active agent disposed within the polymeric matrix, within a coating on the polymeric matrix, or both and (b) one or more hinge elements coupling adjacent modules to form a chain structure. The hinge elements can be configured to: (a) permit bending of the chain in a first direction to enable transurethral insertion; (b) resist bending in a second direction to inhibit expulsion during voiding; and (c) undergo controlled weakening or degradation in a fluid environment. The device can be configured to assume:
a) an insertion configuration, in which the modules are arranged substantially collinearly, having a maximum cross-sectional profile of less than about 10 mm for transurethral administration;
b) a retention configuration, in which at least two modules are oriented non-collinearly with respect to one another by the hinge elements, having a minimum cross-sectional profile of at least about 16 mm in any orientation so as to resist excretion through the urethra; and
c) an elimination configuration, after detachment of one or more modules, in which each individual module has a maximum cross-sectional profile of less than about 6 mm for passive excretion through the urethra; and
   The hinge elements can further configured to permit detachment of the modules, wherein the detachment may be sequential, simultaneous, or a combination thereof.

Embodiments also include an intravesical drug delivery device (“RHL”) that include a first module and a second module. Each module can include (a) a polymeric matrix and (b) at least one active agent disposed within the polymeric matrix. A hinge structure can couple the first module and the second module. A linker can be disposed within or associated with the hinge structure. The linker can be made of a swellable material configured to:
(a) absorb fluid within a bladder environment;
(b) expand in volume in response to the absorbed fluid; and
(c) generate a force sufficient to separate at least one of the first module or the second module from the hinge structure. The device can be configured to (a) be inserted into a bladder in a first configuration, (b) assume a second configuration within the bladder that promotes retention and (c) disassemble into components dimensioned for elimination through a urethra following expansion of the linker.

Embodiments also include a method of manufacturing a modular intravesical drug-delivery device that includes steps of:
a) forming a polymer-drug composition into rod-shaped modules by one or more of hot-melt extrusion, injection molding, compression molding, direct compression, 3D printing, or solvent casting;
b) cutting the rods into modules of variable length;
c) applying a coating via dip-coating, spray-coating, wrapping a pre-formed polymer sheet or film, co-extrusion, placement into a pre-formed polymer sleeve, or solvent-based coating;
d) attaching modules to hinge elements using heat-setting, solvent welding, or mechanical fixation; and
e) ensuring the assembled device maintains drug-uniformity and hinge integrity.

Embodiments also include a method for delivering a therapeutic agent intravesically to a subject. The method can include steps of:
a) inserting the modular device (as described above) into the bladder;
b) allowing buoyant modules to float and distribute drug locally;
c) permitting detachment and elimination of modules over time, whether sequentially, simultaneously, or in any combination, thereby achieving a pharmacokinetic profile linked to device architecture.

Accordingly, embodiments also include an intravesical drug delivery system. The system can include a plurality of particles, each particle comprised of a first cylinder joined to a second cylinder by a hinge. Each of the cylinders can include an excipient portion and an active agent. An interior portion of the hinge can include a linker that swells when exposed to fluid. Upon swelling, the cylinders can separate from the hinge.

In aspects, the linker material swells and pushes out the rods. The linker materials can swell (either due to heat deformation or water absorption) and force the rods out of the sockets. For example, poly(methyl methacrylate), poly(acrylic acid), Carbopol, hydroxypropyl methyl cellulose are types of materials that will swell in solution. The rate of swelling can be controlled via type of hydrophilic swellable polymers, their MW, salt or acetic acid blend, blend of hydrophilic swellable polymers with non-swellable hydrophobic materials, and enteric coatings.

Accordingly, the particles can be attached to one another (i.e., each package includes a first particle joined to a second particle by a hinge). The particles can be administered into the urinary bladder of the subject in a stacked arrangement (i.e., with a small diameter). Once inside the bladder, the particles can conform to an extended arrangement so that they are retained. An active agent can be released during this “retention phase.” Thereafter, individual particles are separated from one another and the components of the particles can be excreted during urination.

In aspects, each of the cylinders has a beta-phase crystalline core and an alpha-phase crystalline shell.

Embodiments also include a method for applying a protective coating or “sleeve” using spray or dip-coating techniques. In aspects, PCL, PCL-GA, PCL-LA or PEG-PCL block copolymers are used to create a flexible and biodegradable layer. The polymer can be dissolved in an organic solvent, ensuring even distribution and uniform adhesion. In aspects, the coating preserves the geometry of the rod in the face of bladder forces that would otherwise break and shorten the rod.

In aspects, the drug products release multiple active agents (e.g., to treat multiple ailments or to target drug-resistant bacteria). In aspects the drug products are homogenous with one another. In aspects, the drug products are not homogenous (e.g., they contain different excipients, have different physical qualities, different active agents and/or different sizes/shapes).

Accordingly, embodiments include systems and methods of treating an ailment such as a urinary system disorder. In aspects, the systems and methods include a pharmaceutical formulation for intravesical administration of an active agent. The formulation can include particles, each having an excipient portion and an active agent. The formulation can be administered by transurethral delivery using a drug delivery device as described herein.

In aspects, particles have a cross-sectional diameter less than or equal to 4 mm for insertion, undergo a conformational change post bladder insertion to a cross-sectional diameter of 10 mm or more for retention, and then reduce to a cross-sectional diameter less than or equal to 4 mm for elimination.

Embodiments also include methods of altering (“tuning”) the particles to determine the retention period. For example, a retention period of about five days can be preferred for a type of treatment. Another type of treatment may require a longer retention period (e.g., about 90 days). In aspects, the linker material is selected based on how much it swells. In aspects, the time of swelling is adjusted (e.g., by varying the area of fluid exposure or applying a coating to the linker material). In aspects, the force necessary to separate the rod from the hinge is adjusted (e.g., by friction, material used, surface characteristics or use of adhesive material).

Embodiments also include methods of delivering a substance or formulation into the bladder of a patient. The methods can include steps of administering a plurality of particles (as described above) into the bladder of a subject and allowing the particles to release a drug from the drug delivery device into the patient's bladder. In aspects, the patient suffers from an ailment of the bladder, urinary tract and/or kidney. The method can also include treating the patient for the ailment. In aspects, the ailment is a urinary tract infection (UTI), bladder cancer, kidney cancer, ureter cancer, urethra cancer, coagulation disease, overactive bladder, underactive bladder, retained urine, diabetes, heart failure, kidney failure or cystitis. The method can also include a step of adjusting the number of particles that are administered based on a desired dose of active agent.

Additional embodiments described herein include methods of treating or preventing urinary tract infection (UTI) by administering formulations and drug products. In aspects, the methods are used with patients who are at risk of UTI and/or have a history of UTIs.

In certain embodiments, the drug product comprises a modular intravesical drug-delivery chain including a plurality of rod-shaped modules connected by one or more hinge elements configured for controlled, staged detachment.

The hinge elements may permit unidirectional bending to facilitate insertion while resisting reverse bending during voiding, thereby reducing premature expulsion.

Drug-release kinetics may be programmable through module-specific drug loading, hinge degradation timing, coating thickness variation, polymer composition, or geometry.

In aspects, the methods include a step of diagnostic imaging. In aspects, the methods include a step of observing a color change in excreted urine over at least a portion of the period of the retention phase (i.e., via addition of a color indicator to the drug product).

### BRIEF DESCRIPTION OF THE DRAWINGS

FIG. 1 depicts a particle in a retention configuration (extended) and its three main components: a linker (104), a hinge (120) and an API embedded cylindrical rod (101) with rounded ends (102). The rods are held together through the hinge with endcaps (103) which hold the linker against the rod.

FIG. 2A depicts the particles in an external environment (i.e., before they are inserted), in a stacked arrangement for administration and in a retention environment (i.e., inside the bladder). The particles are administered using a delivery conduit (105) such as a catheter or a sheath.

FIG. 2B is a sequential illustration that depicts a particle after implantation. Drug delivery occurs as the agent (106) passes from the rods (101) to the fluid environment.

FIG. 3A depicts different mechanisms (i.e., different portions for allowing release of rods) of particle separation according to embodiments. Caps (103) can include pores (108) to allow entry of fluid and gradual separation. Alternatively, the caps (103) can include slits (107). In an alternative design, the caps are thinner or more shallow (111) to allow entry of fluid around a perimeter region.

FIG. 3B depicts an alternative design. The endcap (109) can be made of a biodegradable material to allow particle separation. Alternatively, a hinge connection (110) is biodegradable.

FIG. 4 depicts an embodiment (RHL) in which the cylinders (or a portion) swell upon exposure to liquid to release the rods after a retention period. As shown, a linker (104) inside the caps (103) gradually expands to expel the rods.

FIG. 5A depicts cylindrical rods released from endcaps after the swelling of the linker (104) to occupy the full volume of the endcap or greater than the full volume of the endcap.

FIG. 5B depicts a composition for an expanding pellet and its composition: (112) 2-Octyl-CA, (113) PVP, (114) PAA and (115) coating of 2-Octyl-CA.

FIG. 6 depicts an embodiment in which a polymeric coating is used to allow fluid entry and dispersion of an active agent. A biodegradable coating polymer is depicted intact (116) and after partial degradation (117) to allow fluid ingress and drug release.

FIG. 7 depicts an alternative design (4RC) in which two pairs of cylinders (101) are joined together with a hinge (110). A knee joint (118) joins cylinders (101) into pairs. The cylinders detach from one another after a retention period in liquid. In this design, the rods are encased in a polymer casing (e.g., EVA), and a separate biodegradable polymer element (e.g., PCL-GA) functions as the hinge joining the rod pairs. The casing serves to control drug release and protect against rod breakage, while the hinge undergoes degradation to permit disassembly.

FIG. 8 is an image of a drug product prior to intravesical administration. A linker joins two sets of segments to each other. In this design, (“4RC”) a biodegradable polymer hinge element joins the rod pairs, and disassembly occurs through degradation of the hinge. Individual components (i.e., cylinders) detach from one another after a retention period.

FIG. 9A is an image of a drug product prior to intravesical administration.

FIG. 9B depicts a drug product when inserted into a catheter (105) for intravesical administration.

FIG. 9C depicts a drug product prior to placement in a solution.

FIG. 9D depicts the drug product after the individual segments are separated from one another.

FIG. 10 is a schematic of the phases during the construction of the 4RC product. The process can be divided into six stages. First, rods (101) are formed from the excipient admixed with the API. Next, the rods are coated (116) with a polymer such as EVA. Rods are then cut to the appropriate length and the ends given a rounded geometry (102) that will allow interaction with the bladder wall and urethra without injury. The rods are then joined in-line with a biodegradable polymer adhesive (119) such as PCL-GA to form knee joints (118). Thereafter, the limb pairs are joined with PCL-GA to form the angled hip hinge (110) for retention.

FIG. 11 depicts an in vivo drug release curve of amikacin from intravesical drug products. This experiment was performed in Yucatan minipigs with a single administration of ten 4RC drug products. A total of 750 mg amikacin was delivered into each animal.

FIG. 12A depicts a configuration in which cylinders have different active agents. Here, a first agent (e.g., an antibiotic) is administered with a second complementary agent. For example, amikacin is administered to treat an infection (e.g., UTI) in combination with oxybutynin to treat overactive bladder, as these ailments often coexist.

FIG. 12B depicts a configuration in which cylinders have different polymer compositions. The polymer compositions can determine the rate of release of an agent and the duration of retention. This allows administration of a “burst” or induction phase (e.g., five days) of an agent followed by a maintenance phase (e.g., 45 days).

FIG. 12C depicts a configuration in which the cylinders (of an RHL construct) have different compositions (i.e., polymer compositions and/or agents).

FIG. 13 is a cross-sectional image of a cylindrical rod (101) comprised of an outer alpha-phase crystalline shell (122) and a beta-phase crystalline core (123) for controlled drug release and stability.

FIG. 14A is a graph that shows the minimum temperature required to generate isotropic melting.

FIG. 14B is a graph that shows α-crystallization exotherm.

FIG. 14C is a graph that shows α-melting endotherm.

### LIST OF REFERENCE NUMBERS

The following reference numbers are used in the figures and written description.
101=rod (API embedded cylindrical rod)
102=rounded end of rod
103=cap or endcap
104=linker
105=delivery conduit/catheter
106=drug molecule
107=slits
108=pores
109=cap (biodegradable)
110=hinge or hip connection (biodegradable)
111=cap (shallow design without rod overlap)
112=2-Octyl-CA
113=PVP
114=PAA pellet
115=coating of 2-Octyl-CA
116=intact biodegradable coating
117=coating broken down (allows water entry and drug exit)
118=knee joint
119=knee polymer coating
120=hinge polymer (non-biodegradable)
121=rod coating (biodegradable or non-biodegradable)
122=alpha polymorph shell
123=beta polymorph core

The above-described drawing figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### Definitions

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can be in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

The term “over-active bladder” or “OAB” refers to a group of urinary symptoms (rather than a disease). The most common symptom is a sudden, uncontrolled need or urge to urinate. Some people will leak urine when they feel this urge. Another symptom is the need to pass urine many times during the day and night. OAB can be described as a constant urge to urgently urinate. Leaking urine is called “incontinence.”

The term “stress urinary incontinence” or “SUI” refers to another common bladder problem. Subjects with SUI leak urine while sneezing, laughing or doing other physical activities.

The term “interstitial cystitis,” “IC” or “bladder pain syndrome” refers to a chronic, or long-lasting, condition that causes painful urinary symptoms. Symptoms of IC can be different from person to person. For example, some people feel mild discomfort, pressure, or tenderness in the pelvic area. Others may have intense pain in the bladder or struggle with urinary urgency, the sudden need to urinate, or frequency, the need to urinate more often. Health care professionals diagnose IC by ruling out other conditions with similar symptoms.

The term “GAG layer restoratives” refers to therapeutic agents (e.g., hyaluronic acid, chondroitin sulfate, and heparin) used to repair the protective glycosaminoglycan lining of the bladder. Primarily used for chronic cystitis, bladder pain syndrome (BPS/IC), and recurrent UTIs, these treatments are most effective when delivered using intravesical particles (i.e., directly into the bladder) to seal the urothelium against toxins, reducing inflammation and pain.

The term “non-muscle invasive bladder cancer” or “NMIBC” refers to a type of cancer found in the tissue lining of the bladder that has not spread into the deeper, muscular bladder wall. It represents about 70% of bladder cancers, is highly treatable via surgery, but has a high rate of recurrence. Symptoms include blood in the urine (hematuria), urgent or frequent urination, and burning sensations. Conventional treatment includes a transurethral resection of the bladder tumor (TURBT), wherein the tumor is removed through the urethra. This is often followed by intravesical therapies (medication placed directly into the bladder), such as BCG immunotherapy.

The term “BCG” or “Bacille Calmette-Guerin” refers to a form of immunotherapy that is a treatment for non-muscle invasive bladder cancer (NMIBC) that uses a weakened form of Mycobacterium bovis bacteria, similar to the tuberculosis vaccine, to trigger the immune system to attack cancer cells. It is administered directly into the bladder via a catheter (intravesical therapy) to prevent recurrence and progression. However, many patients do not respond to BCG, and BCG treatment can also induce a range of adverse effects leading to discontinuation of treatment. Chemotherapeutic agents are usually reserved for patients who have failed BCG therapy. Chemotherapy can be administered intravesically to concentrate the chemotherapeutic agent at the tumor sites and eliminate any residual tumor after resection while avoiding systemic exposure of the drug.

The term “muscle-invasive bladder cancer” or “MIBC” refers to cancer that spreads into the detrusor muscle of the bladder wall, representing a more aggressive stage (usually Stage II or higher) than non-muscle invasive types. It typically requires intensive treatment, such as bladder removal (cystectomy) or bladder-sparing approaches like chemo-radiotherapy.

The term “induction phase” refers to the initial, intensive phase of cancer treatment, commonly used for leukemias and some solid tumors, designed to rapidly destroy as many cancer cells as possible and induce remission. It is the first step in a treatment regimen, often aiming to bring the disease under control before consolidation or maintenance therapy.

The term “catheter” refers to a flexible tube that can be inserted through several points of entry and into a patient or subject's body cavity, duct, vessel, brain, skin or adipose tissue. Catheters perform several functions including allowing drainage of body fluids, administration of substances and materials into a patient or subject and enabling access by surgical instruments to a patient or subject.

The term “Foley catheter” refers to a flexible tube that a clinician passes through the urethra and into the bladder to drain urine.

An “indwelling catheter” is a catheter typically retained in a fixed position by means of an inflated balloon attached to the outer body of the catheter.

A “vesical sheath” refers to a hollow tube which functions as a conduit for introducing surgical instruments, substances, and other media to the urinary system of a subject or patient. As used herein, the sheath refers to the middle section of the device between the distal balloon tip and the proximal lock ring assembly. The main length of the primary lumen is in the middle sheath portion, but the primary lumen is open from end to end for delivery of product through the entire device. The sheath should have low friction on the outside and in the primary lumen so that it can be installed with minimal distress to the patient and so that the product can slide through it easily. Materials can be, for example, silicone coated fibers, spring, or struts (for a combination of strength and flexibility in a thin material), coating with PTFE for low friction, or metal for strength in a thin material.

The term “active agent,” “active ingredient” or “active pharmaceutical ingredient” or “API” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An active agent can be a primary active agent, or in other words, the component(s) of a composition to which the whole or part of the effect of the composition is attributed. An active agent can be a secondary agent, or in other words, the component(s) of a composition to which an additional part and/or other effect of the composition is attributed.

The term “drug product” refers to any substance, device, particle or product suitable for delivery into a subject or patient for the treatment of a disease, condition, malady or disorder, and may further include excipients, adjuvants, carriers, placebos, powders, foams, gels, liquids, capsules, coatings, time-release mechanisms, delayed-release mechanisms, robotic medicaments, computer-programmed drug products, products driven or guided by artificial intelligence or machine learning, transmitters, or any substance, device or product known in the art that is suitable for delivery to a patient or subject.

The term “pharmaceutically acceptable” is meant a material that is not biologically or otherwise undesirable, i.e., the material may be incorporated into a pharmaceutical composition as provided herein and not cause any substantial undesirable biological effects or interact in a deleterious manner with any of the other components of the composition. When the term “pharmaceutically acceptable” is used to refer to a solid or semi-solid carrier, liquid vehicle, or other excipient, i.e., to any inactive ingredient herein, it is implied that the carrier or excipient has met the required standards of toxicological and manufacturing testing and/or that it is included on the Inactive Ingredient Guide prepared by the U.S. Food and Drug Administration and designated “Generally Regarded as Safe” (“GRAS”).

The term “controlled-release” or “CR” refers to a medication, carrier or other platform that releases an active ingredient at a specific rate to keep constant medication levels in a patient's body for a specific time period. This allows medication levels to remain at an effective level for a duration of time. Unlike sustained release (SR) forms, CR medications can precisely control the amount of medication in the body at a given time, not just its release. Administration of a controlled-release active agent-containing formulation or fraction thereof does not result in the immediate release of 100% of the active agent. The term is used interchangeably with “nonimmediate release” as defined in Remington: The Science and Practice of Pharmacy, Nineteenth Ed. (Easton, PA: Mack Publishing Company, 1995). In general, the term “controlled release” as used herein includes sustained release, modified release, pulsatile release, and delayed release formulations, as well as formulations that combine two or more types of release profiles, such as immediate release of a bolus dose followed by pulsatile release or sustained release thereafter. “Sustained release” (synonymous with “extended release”) refers to a formulation that provides for gradual release of an active agent over an extended period of time, and that preferably, although not necessarily, results in substantially constant levels of an agent in a volume of distribution (e.g., urine, bladder, total body water, etc.), and is normally referred to as “zero order” release. “Controlled release” also includes “delayed release,” indicating a formulation that, following administration to a patient, provides for a measurable time delay before the active agent is released from the formulation into the patient's body. Controlled release dosage forms herein, however, are generally of the sustained release dosage type.

The term “particles” or “microparticles” refers to a carrier or controlled release carrier described herein that is formulated so as to contain a pharmacologically active agent. As used herein, a particle can also refer to a pair of cylindrical products attached by a linker. The particles can be substantially spherical, cylindrical or rod-shaped or have some other shape. Particle size is given as the mean particle diameter in a population of particles. The particle size distribution in a population of particles is relatively narrow (i.e., the specified mean particle size is associated with a fairly low standard of deviation, typically less than 30%), for example, less than 20% or less than 15%. Particles within a formulation may or may not have substantially the same shape and size. The terms “particles” and “microparticles” may be used interchangeably with the term “drug product” and is also a form of drug product with the term drug product being utilized as an umbrella term that includes both particles and microparticles.

The terms “intravesical delivery particle grouping,” “solid drug product grouping,” “drug product capsule grouping,” “drug product capsule grouping,” “delivery particle grouping,” “coupled drug product grouping,” “connected drug product grouping,” “drug product grouping,” “hinged drug product grouping” or “particle grouping” are interchangeable and refer to a connection of separate drug particles or drug products or structures via a tether or other equivalent connective structure.

The term “intravesical administration” or “intravesical therapy” refers to a treatment wherein a therapeutic is put directly into the bladder (i.e., through a catheter or similar device) rather than being given orally or injected into a vein. Similarly, an intravesical drug is directly administered into the urinary bladder via a urethral catheter.

The term “enteric coating” refers to a polymer barrier that prevents its dissolution or disintegration in solution. As used herein, a cylinder pellet made of a swellable material can be coated in a slow-degrading adhesive material. This can be placed in the socket that joins the cylinders. In this design, the adhesive coating helps keep the cylinders together while the drug payload is delivered. Thereafter, as the coating degraded/dissolves over time, water can diffuse through accessing the swellable material cause swelling of the material (e.g., to 10× size) which pushes out the rod. In aspects, the rate of swelling is adjusted by changing the rate that water diffuses in the socket by modifying the silicone hinge. This can include, for example, a) changing length of the hinge and its thickness, b) slits on the side and/or c) holes of varying diameter on the caps of the hinge and/or combinations.

The term “hinge element” or “connection element” as used herein refers broadly to any structural feature, material, coating, bond, or mechanism that (a) joins two or more adjacent modules to one another, thereby maintaining the modules in a connected configuration, and (b) is configured to permit eventual separation of the modules from one another, whether by chemical degradation, hydrolytic erosion, enzymatic cleavage, dissolution, swelling-induced displacement, mechanical fatigue, loss of adhesion, or any other mechanism. As used herein, the term “hinge element” is not limited to a mechanical hinge having a defined bending axis, but encompasses any connection between adjacent modules that maintains structural continuity during a retention phase and thereafter permits disassembly. Non-limiting examples of hinge elements include: a discrete molded or formed hinge body connecting two modules; a polymeric coating applied over the junction between adjacent modules that degrades or erodes over time; a dissolvable or degradable adhesive bond between adjacent modules; a degradable polymeric wrap, sleeve, or band encircling two or more adjacent modules; a filament, suture, or thread passing through or connecting adjacent modules; a mechanically interlocking joint configured to release under predetermined conditions; and a swellable linker material disposed within a socket or cavity between modules that expands upon fluid exposure to displace the modules from one another. The terms “hinge,” “hinge element,” and “connection element” are used interchangeably throughout this specification unless otherwise indicated.

The term “spherical” herein refers to a precisely spherical particle or, more generally, to a particle or object with a rounded surface that may or may not be substantially spherical.

The terms “mean diameter,” “mean longest dimension,” and “particle size” are used interchangeably herein. As used herein, the particle size is given by mean diameter for spherical particles and mean longest dimension for non-spherical particles. It should be noted that the term “mean diameter” technically corresponds to substantially spherical particles; however, other particle shapes can also be incorporated into the particulate formulation, providing that the particles have at least one dimension (i.e., at least one mean dimension, in the overall population of particles) greater than 2.0 mm, e.g., in the range of 2.0 mm to about 12.0 mm, e.g., about 2.5 mm to about 6.5 mm, e.g., about 2.5 mm to about 6.0 mm, about 2.5 mm to about 5.5 mm, about 2.5 mm to 5.0 mm, etc., as before. An elongated particle, e.g., a cylindrical or rod-shaped particle, could, for example, have a length of about 9.0 mm and yet be narrow enough to pass through a urethral catheter and the urethral opening.

The terms “effective amount” and “therapeutically effective amount” of an active agent, an active agent combination, or a pharmaceutical formulation refer to an amount or concentration that is nontoxic but sufficient for producing a desired result. The exact amount required will vary from subject to subject, depending on factors such as the age, weight and general condition of the subject, the particular condition being treated, the severity of the condition, the specific active agent, and the judgment of the clinician.

The term “urinary system” is used in the conventional sense to refer to the lower urinary tract as well as the upper urinary tract, and thus includes the bladder, urethra, kidneys, and ureters.

The term “disorder” refers to a physiological condition of clinical relevance and thus includes symptomatic or asymptomatic conditions regardless of etiology. Disorders thus include adverse conditions resulting from disease or injury. The disorders addressed with the present invention are disorders of the urinary system.

A “drug delivery time period” refers to a period of time during which a pharmacologically active agent is released from a formulation or fraction thereof, generally an extended time period.

The term “subject” or “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutically acceptable composition” as used herein refers to a composition comprising at least one compound as disclosed herein formulated together with one or more pharmaceutically acceptable carriers.

The term “treating” or “treatment” refers to one or more of (1) inhibiting the disease; e.g., inhibiting a disease, condition or disorder in an individual who is experiencing or displaying the pathology or symptomatology of the disease, condition or disorder (i.e. arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

The term “specific gravity” refers to the ratio of a material's density with that of water at 4° C. It is therefore a relative quantity with no units. The specific gravity of urine refers to the electrolytes and urine osmolality. The normal range for urine specific gravity is 1.005 to 1.030. Specific gravity, in the context of clinical pathology, is a urinalysis parameter commonly used in the evaluation of kidney function and can aid in the diagnosis of various renal diseases.

The term “glyceryl tripalmitate” or “GTP” refers to a triacylglycerol with the formula C₅₁H₉₈O₆, structured as three 16-carbon palmitic acid chains each esterified to one of the three hydroxyl groups of glycerol. It is a hydrophobic material with a density less than 1. It exists in several crystalline forms. Low melting allows for ease of manufacturing final drug product. Alternatives to GTP include other fatty acid of glycerol including glyceryl tristearate (also known as tristearin used in multiple injectable products).

The term “poly(ethylene-co-vinyl acetate)” or “EVA” refers to a versatile polymer that can be modified by MW and most notably by the ratio of hydrophobic ethylene to hydrophilic acetate.

The term “poly(caprolactone-co-glycolide)” or “PCL-GA” refers to a versatile polymer that can be elastomeric to fluid depending on composition and molecular weight.

The term “polycaprolactone” or “PCL” refers to a biodegradable, biocompatible polyester polymer, synthesized through ring-opening polymerization of ε-caprolactone, used in various applications including drug delivery, tissue engineering, and implantable devices due to its slow degradation rate and ease of manipulation.

The term “poly(lactide-co-glycolide)” or “PLGA” refers to another biodegradable polymer, known for its faster degradation rate compared to PCL. PLGA has a higher intrinsic mechanical stiffness and strength than PCL. Blends of PCL and PLGA can combine the benefits of both polymers, such as controlled drug release and tunable mechanical properties.

As used herein, the term “biodegradable” refers to a material that undergoes chemical degradation (e.g., hydrolysis, enzymatic cleavage, or oxidative breakdown) under physiological conditions over a clinically relevant time period, resulting in fragments or byproducts that are cleared from the body through natural metabolic pathways. The terms “bioresorbable” and “bioabsorbable” are used herein to refer to materials that are similarly broken down and assimilated or eliminated by the body, and unless otherwise indicated, these terms are to be understood as encompassed within the scope of “biodegradable” as used in this specification and the claims appended hereto. Accordingly, any embodiment described herein as comprising a biodegradable material is understood to also encompass embodiments in which the material is bioresorbable or bioabsorbable, and vice versa. For the avoidance of doubt, the claims reciting “biodegradable” materials are intended to cover materials that degrade, are resorbed, or are absorbed by the body, regardless of the specific mechanism by which the material is broken down and eliminated.

The term “2-octyl cyanoacrylate” or “2-octyl CA” refers to a cyanoacrylate ester typically used as a wound closure adhesive. It is closely related to octyl cyanoacrylate.

The term “polyvinylpyrrolidone” or “PVP” also commonly called polyvidone or povidone, refers to a water-soluble polymer compound made from the monomer N-vinylpyrrolidone. PVP is available in a range of molecular weights and related viscosities, and can be selected according to the desired application properties.

The term “poly(acrylic acid)” or “PAA” refers to a synthetic, high-molecular-weight polymer derived from acrylic acid, known for its anionic nature and ability to absorb and retain water, making it useful as a superabsorbent, thickener, and dispersing agent in various applications.

The term “hygroscopic material” refers to a substance that absorbs water and expands. Many engineering polymers are hygroscopic, including nylon, ABS, polycarbonate, cellulose, carboxymethyl cellulose, and poly(methyl methacrylate) (PMMA, plexiglas, perspex).

The term “swelling” refers to the change in volume of a substance as it absorbs a compatible fluid (e.g., water or urine). As the fluid is absorbed it causes changes in shape and/or volume. Similarly, the term “swellable” refers to a substance that is capable of or tending to swell (i.e., to increase in size or volume) usually by absorbing liquid or pressure. A “swellable substance” refers to something that has the ability to become larger or expand. As used herein, this swelling is caused by the absorption of fluids or the application of pressure, causing the material (i.e., linker) to expand.

The term “alpha-phase crystalline” refers to a crystalline portion of a material in which the constituent molecules are arranged in the α-polymorphic form, the α-form being a distinct crystal structure that is differentiable from other polymorphs (such as β-, γ-, or δ-forms) by its characteristic lattice geometry, molecular conformation, X-ray diffraction pattern, spectroscopic features, and/or thermal transitions. As used herein, “alpha-phase crystalline” encompasses any region of a material that exhibits the structural attributes of the α-phase, regardless of the processing conditions or methods used to generate that phase.

The term “beta-phase crystalline” refers to a crystalline portion of a material in which the constituent molecules are arranged in the β-polymorphic form, the β-form being a distinct crystal structure that is differentiable from other polymorphs (including, for example, α-, γ-, or δ-forms) by its characteristic lattice parameters, molecular conformation, spectroscopic signature, and/or thermal behavior. As used herein, “beta-phase crystalline” encompasses any region of the material whose crystal structure exhibits the defining structural features of the β-phase, regardless of how that phase is produced (e.g., by mechanical, thermal, chemical, or nucleating processes). For example, the beta phase is found in polylactic acid (PLA) and isotactic polypropylene.

The term “dip coating” refers to a technique where a substrate is submerged in a coating slurry, then withdrawn, allowing the excess coating to drain off, leaving a uniform thin film.

The term “amikacin” refers to an antibiotic medication used for a number of bacterial infections. This includes joint infections, intra-abdominal infections, meningitis, pneumonia, sepsis, and urinary tract infections (UTIs). It is also used for the treatment of multidrug-resistant tuberculosis. It is typically used by injection into a vein using an IV or into a muscle.

The term “excipient” refers to pharmacologically inert ingredients added intentionally to a drug product for various functional roles, such as to enhance dosage form volume or size, disintegration of solid dosage forms, binding of particulates, lubrication during processing, taste masking or modifying drug release.

The term “zero-order” refers to a delivery system that releases a drug at a constant rate (i.e., the release rate is independent of the concentration or the amount of drug that remains in the delivery system). Drug release kinetics is said to be zero-order kinetics when a constant amount of drug is eliminated per unit time but the rate is independent of the concentration of the drug. Zero-order drug delivery systems (DDS) can overcome the issues faced by immediate-release and first-order systems by releasing the drug at a constant rate, thereby maintaining drug concentrations within the therapeutic window for an extended period. In aspects, the drug products described herein are configured for zero-order release of an active agent.

The term “first-order” refers to a drug release rate that is directly proportional to the concentration gradient and is a function of the amount of drug remaining in the dosage form. Similarly, “sustained release” refers to the slow release of a drug over an extended period after administration of a single dose. In aspects, the drug products described herein are configured for first-order release of an active agent.

The terms “therapeutic index,” “TI” or “therapeutic ratio” refer to a quantitative measurement of the relative safety of a drug. It is a comparison of the amount of a therapeutic agent that causes the therapeutic effect to the amount that causes toxicity. A therapeutic window or safety window refers to the range of doses that optimize between efficacy and toxicity, achieving the greatest therapeutic benefit without resulting in unacceptable side effects or toxicity. TI is calculated from the ratio of the dose of a drug that causes adverse effects at an incidence/severity not compatible with the targeted indication (e.g., toxic dose in 50% of subjects, TD₅₀) to the dose that leads to the desired pharmacological effect (e.g., efficacious dose in 50% of subjects, ED₅₀).

The term “substantially” indicates the possibility of slight deviation from a recited chemical or physical property and allows for a difference of at most about 20%, or at most about 10%, or at most about 5%, between an actual chemical or physical property and the recited chemical or physical property. The term “substantially homogeneous,” for example, refers to a material in the form of a mixture of two or more components in which the material is substantially uniform throughout, with any two discrete regions within the material differing by at most about 20%, or at most about 10%, or at most about 5%, with respect to a chemical or physical property of the material, such as the presence or absence of a component, the concentration of a component, the degree of hydrophilicity or lipophilicity, density, crystallinity, or the like. Similarly, the term “approximately” or “about” in any context is intended to connote a possible variation of at most about 20%. Generally, the term connotes a possible variation of at most about 10%, or at most about 5%.

Other technical terms used herein have their ordinary meaning in the art that they are used, as exemplified by a variety of technical dictionaries. The particular values and configurations discussed in these non-limiting examples can be varied and are cited merely to illustrate at least one embodiment and are not intended to limit the scope thereof.

### DETAILED DESCRIPTION

It is to be understood that both the foregoing general description and the following detailed description are exemplary and explanatory and are intended to provide further explanation of the subject technology as claimed. Additional features and advantages of the subject technology are set forth in the description below, and in part will be apparent from the description, or may be learned by practice of the subject technology. The advantages of the subject technology will be realized and attained by the structure particularly pointed out in the written description and claims hereof.

Embodiments include a system of small particles that are relatively easy to administer into the bladder through the urethra. Thereafter, the particles remain in the bladder during a retention period while an agent is released. After the retention period, the particles are excreted in the urine (i.e., passively). The particles can carry an agent such as, for example, an antibiotic, an anesthetic, an analgesic agent, a diuretic, an anti-inflammatory agent, a coagulant or an anti-coagulant, a chemotherapeutic agent, etc.

There are obvious benefits to this approach of drug delivery. When administered to the bladder, “intravesical particles” or “drug products” can deliver drugs locally to the bladder/urethra. This avoids the need for oral medication and systemic administration. Low doses of antibiotics are effective and consequently there are fewer side effects. An improved therapeutic window is also possible with controlled drug release. As described below, a formulation that includes active-agent-containing particles can be administered to a subject via the intravesical route (i.e., through the urethra or via a bladder injection) using a catheter or other device as described herein. Moreover, with passive removal, the implant and/or its parts degrade or dissolve via hydrolysis (i.e., the breakdown of chemical bonds by water), or disassemble gradually, eventually passing naturally with urine. Thus, the patient does not have to return to the clinic for a procedure to remove the particles.

Also described herein are methods of treatment that include administering a drug product into the bladder of a subject. The methods can include intravesical administration of particles using a drug delivery device (e.g., a catheter). Thereafter, the particles can release a therapeutic agent (or multiple agents) into the bladder for a desired course of treatment. The methods are useful for treating ailments such as infections (e.g., UTI), interstitial cystitis, overactive bladder syndrome, bladder cancer, kidney cancer, ureter cancer, urethra cancer, coagulation disease, overactive bladder, underactive bladder, retained urine, diabetes, heart failure, kidney failure or cystitis.

The drug products described herein can be administered directly into the bladder through the urethra (i.e., transurethral administration). In aspects, the drug products are retained in the bladder for an intended duration of time (i.e., a retention period). As used herein, the retention period refers to a duration of at least 24 hours and up to 90 days.

The selection of polymers for the particles can be selected based on the desired duration period. During the retention period, one or more active agents can be released into the bladder and urinary system. After the retention period, the products can be expelled from the bladder by urination (i.e., elimination phase). As can be appreciated, composition of the particles, the size of the particles and diameter of the urethra are relevant to each of these phases.

Though there is variation among humans, the urethra has a normal cross-sectional diameter of about 6 mm with a maximum diameter of about 10 mm. Applicants have demonstrated that a 4 mm object (in diameter) can pass the urethra with minimal irritation. Whereas objects of 5 mm diameter or greater typically cause discomfort and/or complications when expelled. Therefore, Applicants propose that the optimum cross-sectional diameter of an object introduced into the bladder through the urethra is less than or equal to 4 mm. In aspects, the objects are cylindrically shaped with a diameter of about 4 mm.

From human experiments, Applicants have demonstrated that a 4 mm cross-sectional object (D) is not retained in the bladder. Rather, it is voided in the urine with a short (or negligible) retention phase. A 5 mm object can have a longer retention period in the bladder but is also expelled. Reviewing objects in the bladder that are not easily expelled indicates a 16 mm cross-sectional diameter cutoff for retention. Though drug products of this size have a long retention phase, they are not easily administered and must be removed surgically or significantly degraded within the bladder.

It is also worth considering that objects in the bladder are rotated into their minimum cross-sectional diameter by the mechanics of voiding when there is a gradient of flow velocity towards the bladder outlet (i.e., urethra). This means that taking an object that has a cross-sectional diameter (D) of 4 mm and increasing its length (L) (e.g., to 10 mm, 20 mm or 30 mm) has minimal effect on its rate of retention. Because of fluid dynamics, the urethra “sees” it is a 4 mm object during the voiding phase. Hence, drug products of 4 mm diameter with lengths of 10 mm have similar properties to drug products with longer lengths (e.g., 4 mm×30 mm).

### Intravesical Drug Products

Applicants describe herein drug products that are easily administered (i.e., through the urethra), retained in the bladder and subsequently excreted when the bladder is voided. In embodiments, the drug products undergo one or more conformational changes. In aspects, the drug product that is relatively small for insertion, larger for retention and small again for elimination. This allows the products to be administered when small and expand so that they are retained in the bladder for a desired period of time (i.e., a retention period). In aspects, particles have a cross-sectional diameter less than or equal to about 4 mm for insertion, undergo a conformational change post bladder insertion to a cross-sectional diameter of 10 mm or more for retention, and then reduce to a cross-sectional diameter less than or equal to 4 mm for elimination.

### Three Geometric Configurations: Insertion, Retention, and Clearance

In embodiments, the drug products described herein are configured to assume three distinct geometric configurations over the course of their functional lifetime: an insertion configuration, a retention configuration, and a clearance (elimination) configuration. Each configuration is defined by the effective cross-sectional profile of the device or its components and is engineered to satisfy the corresponding functional requirement of that phase.

The insertion configuration is the geometry assumed by the drug product during transurethral administration. In this configuration, the modules are arranged substantially collinearly (i.e., in-line) so that the drug product can be loaded into a catheter or delivery device and advanced through the urethra. Because the human urethra has a maximum dilatable diameter of approximately 10 mm, the drug product in the insertion configuration has a maximum cross-sectional profile of less than about 10 mm. In preferred aspects, the maximum cross-sectional profile during insertion is less than or equal to about 5 mm, and in further preferred aspects, less than or equal to about 4 mm. A smaller insertion profile reduces patient discomfort and the risk of urethral injury during administration.

The retention configuration is the geometry assumed by the drug product after it has been deployed within the bladder and before disassembly has occurred. In this configuration, the hinge elements position at least two modules (or limbs) of the drug product in a non-collinear orientation with respect to one another—for example, substantially orthogonal (i.e., approximately 90 degrees) to one another. The purpose of this non-collinear arrangement is to ensure that the minimum cross-sectional profile of the drug product, in any orientation as the device tumbles freely within the bladder, exceeds a retention threshold sufficient to prevent the device from being expelled through the urethra during voiding. Because objects in the bladder are hydrodynamically rotated into their minimum cross-sectional diameter by the flow velocity gradient toward the bladder outlet during voiding, it is the minimum cross-sectional profile across all possible orientations that determines whether the device will be retained. In aspects, the retention configuration has a minimum cross-sectional profile of at least about 16 mm in any orientation. In further aspects, the minimum cross-sectional profile is at least about 18 mm, at least about 20 mm, at least about 22 mm, or at least about 24 mm. For example, a drug product comprising four rod-shaped modules, each having a diameter of about 4 mm and a length of about 10 mm, arranged in two orthogonal pairs by hinge elements, can achieve a retention profile of approximately 24 mm. The retention threshold of approximately 16 mm represents the minimum effective diameter, as determined by Applicants, below which the risk of premature excretion through the urethra becomes clinically significant. In aspects, the retention configuration produces a cross-sectional profile sufficient to prevent passage through a human urethra under physiological voiding pressures.

The clearance configuration (also referred to herein as the elimination configuration) is the geometry of the individual components of the drug product after all connections between modules have been lost (i.e., after complete disassembly). In this configuration, each individual module or component has a maximum cross-sectional profile small enough to be passively excreted through the urethra during normal voiding without discomfort or medical intervention. In aspects, each individual module in the clearance configuration has a maximum cross-sectional profile of less than about 6 mm. In preferred aspects, each individual module has a maximum cross-sectional profile of less than or equal to about 4 mm. As Applicants have demonstrated, objects with a cross-sectional diameter of 4 mm or less pass the urethra with minimal irritation, whereas objects of 5 mm or greater typically cause discomfort and/or complications during expulsion. Accordingly, the drug product is engineered so that, upon complete disassembly, no component remains that has a cross-sectional profile large enough to require surgical removal or to cause urethral obstruction.

### RHL Drug Product

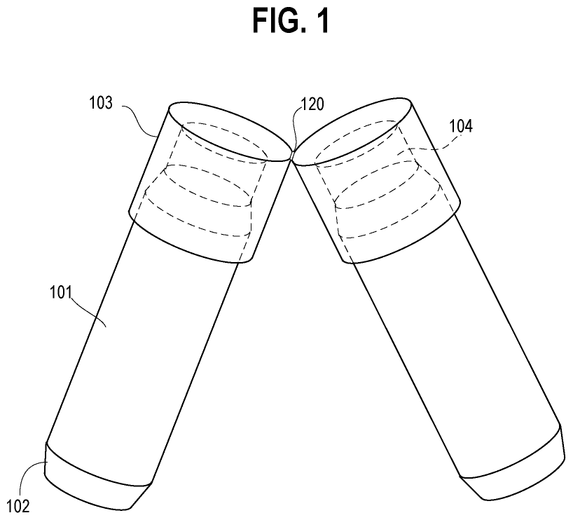

FIG. 1 depicts a drug product (“particle”) in a retention configuration (extended) and its three main components (i.e., a linker, a hinge and an API embedded cylindrical rod). The drug product (“RHL complex”) includes three main components:
Linker—which joins the rod with the hinge and is dissolvable or degradable via hydrolysis, or swellable or biodegradable material;
Rod or cylinder—API embedded in a polymer matrix;
Hinge—silicone (or other material) that connects rods with one another for retention. As shown, the hinge includes 1) a socket (where rod is inserted), 2) a cap (flat round top of socket) and 3) a connector (flexible link or joint that bridges two caps).
   In aspects, each cylinder is secured to a cap. A bridge portion is linked to a connection. One or more of these components can degrade.

In aspects, the size of the intact drug product is resistant to excretion from the bladder (e.g., 10 mm or larger). The individual components are sufficiently small (e.g., 4 mm or smaller) so that they are readily excreted in the urine.

As noted, the hinge can be flexible to allow the drug product to be placed into a syringe for insertion into the bladder. Thereafter, a first conformational change (post-insertion) occurs mechanically through a bend or articulation of a connector between each side of the hinge. The particle resumes the retention configuration. A second conformational change occurs inside the bladder through expansion or swelling or a portion of the linker. The individual components can then be excreted in the urine.

In aspects, the first conformational change (e.g., from 5.5 mm to 24 mm) occurs after insertion and before the first voiding event (i.e., the first urination). In aspects, the second conformational change (e.g., from 24 mm to 5.5 mm or less) occurs after the course of treatment (i.e., drug delivery) or gradually during the course of treatment. This allows the drug products to be retained for the term of a drug treatment (i.e., during administration of an active agent). As can be appreciated, the drug products can include materials known to expand when exposed to a liquid (i.e., hygroscopic materials).

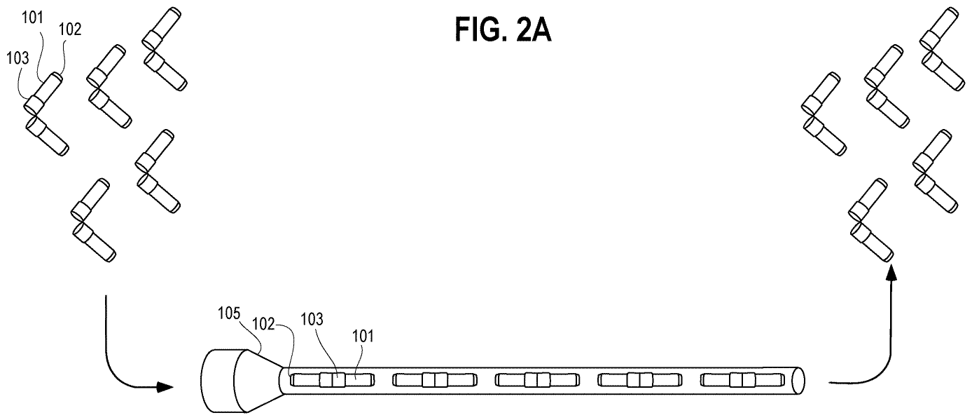

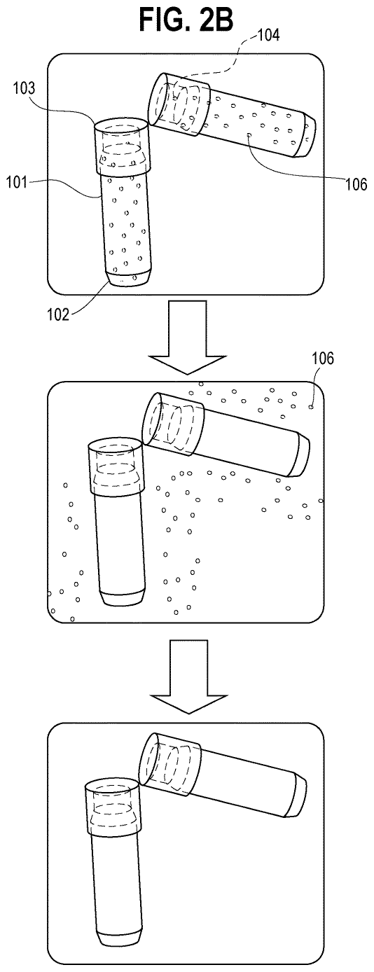

FIGS. 2A and 2B depict the steps of implantation, drug delivery and product disassembly. The drug product (i.e., particles) can be administered into the urinary bladder of the subject in a stacked arrangement (FIG. 2A). As shown, the particles are aligned with one another and inserted through a catheter with a sheath. Thereafter, the particles are retained as the cylinders revert to staggered arrangement (i.e., at an angle to each other in the retention environment). As shown in FIG. 2B, an active agent (i.e., payload) is released during this retention phase. Material in the cap region swells upon exposure to urine in the bladder. This causes the release of the cylinders from the cap. Upon release from one another, the individual components are sufficiently small so that they are excreted during urination.

Other mechanisms of product disassembly are contemplated. For example, one or both of the caps can degrade or dissolve to release the cylindrical particles from one another. Alternatively, the bridge portion degrades or dissolves to release the cylindrical particles from one another. In yet another embodiment, one or both of the cylinders degrade or dissolve.

As can be appreciated, the size/dimensions of the particles are significant. The dimensions FIG. 1 depicts a drug product in a retention configuration and three components (i.e., a linker, a hinge and an API embedded cylindrical rod). As shown, the free end of each cylinder is about 4 mm in diameter. The capped portion is about 5 mm in diameter. As described above, degradation of one or more of the components (i.e., linker, hinge or cylindrical rod) can trigger release of the cylinders from each other. In aspects, the linker is dissolvable, degradable or biodegradable material; the hinge includes a silicone connecting piece that connects to rods and improves retention in the bladder; and the rod is embedded in a polymer matrix. In aspects, the hinge can further include a) a socket (where rod is inserted), b) a cap (flat round top of socket) and c) a connector (flexible link or joint that bridges two caps).

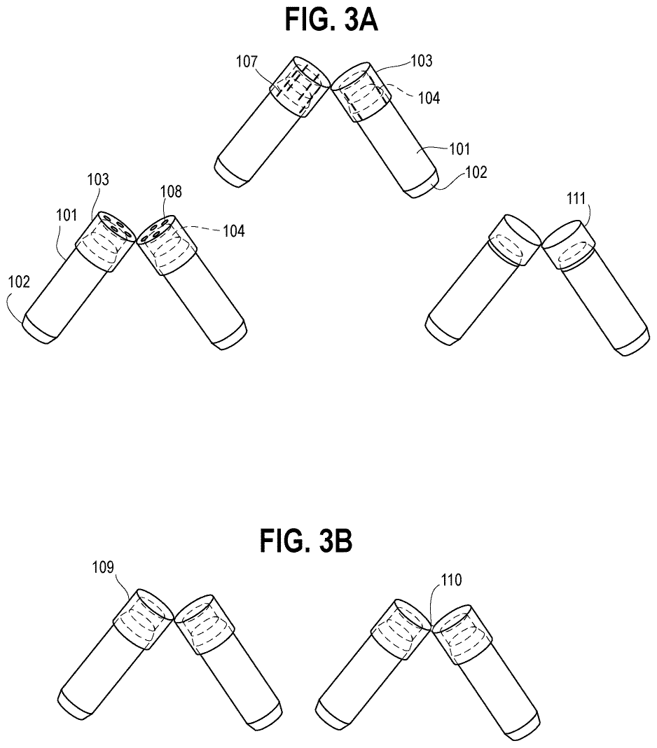

FIG. 3A depicts different mechanisms of particle separation according to embodiments. In aspects, pores on the hinges allow water to enter. The cylinders gradually swell until they are released. In aspects, slits on the hinges increase freedom of movement. The cylinders are released as adhesive material loses its strength. In aspects, adhesive material in a butt joint connects the cylinders. As the adhesive material dissolves, the cylinders are released.

Similarly, FIG. 3B depicts different mechanisms of particle separation according to embodiments. In aspects, the drug products include a “dissolve hinge.” Rather than use hinges made from silicone material, a biodegradable elastomer is used. This material gradually dissolves to release the cylinders. Alternatively, the drug products have a “rod with hinge disassemble.” An erodible material is used to link the connectors. The cylinders disassemble (i.e., separate) upon socket degradation.

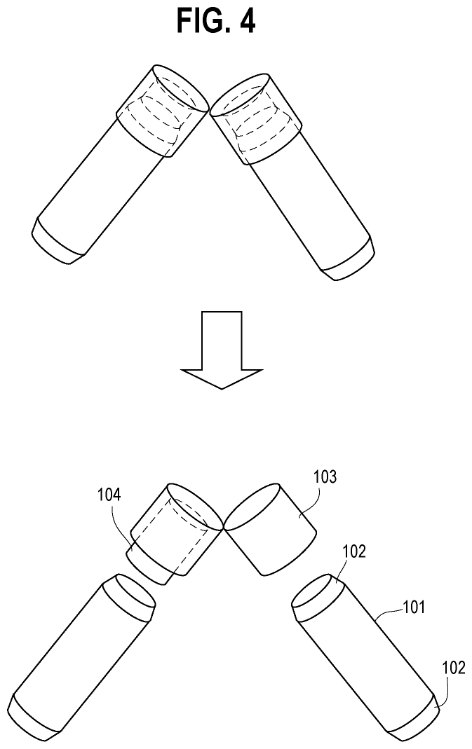

FIG. 4 depicts the disassembly of the components. After a retention phase, material at the hinge region of the drug particles expands and pushes the rods out of the hinge. The individual components are separated from one another to be excreted during urination. The composition/materials can be modified to extend the duration of the retention phase (i.e., the cylinders and cap assembly can be modified or “tuned” to extend the retention phase).

In embodiments, the drug products are passively expelled from the bladder in the urine. In aspects, they dissolve/degrade to a smaller size or pieces so that the pieces can be naturally expelled through the urethra during urination. Alternatively, the rods can be coated with a degradable surface coating such that when the coating dissolves, the rod no longer fit in the socket and fall off thereby causing disassembly of the embodiment. Alternatively, the silicone material of the hinge can be replaced with a biodegradable elastomer that is just as flexible and elastic as silicone and dissolves over time.

As can be appreciated, the area that swells can be composed of different material. In aspects, a blend of materials is used. A mix of “swellable” material with “non-swellable” or hydrophobic material can be used to delay retention period. The ratio can be adjusted based on the desired duration of the retention period.

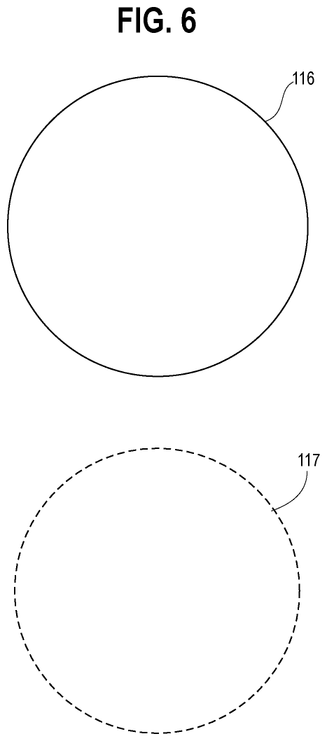

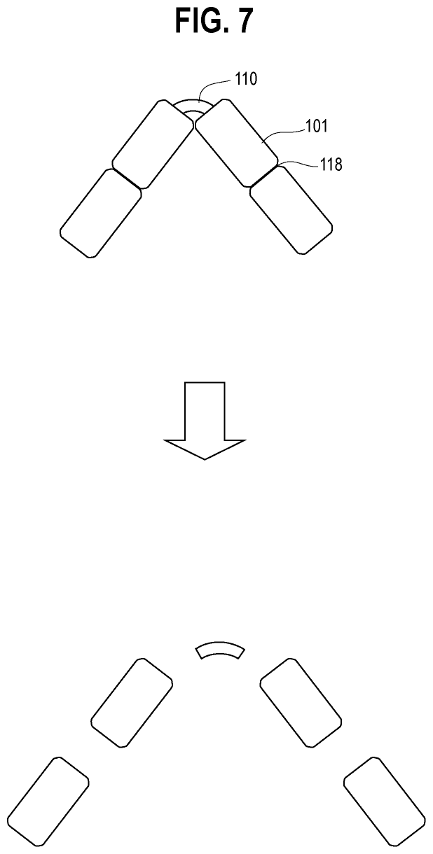

The “swellable” material can also be coated to extend the retention period. As shown in FIG. 6, an enteric coating can be used. The outer layer can reduce water movement/absorption to the swellable material. The coating gradually dissolves upon exposure to urine in the bladder.

A cylinder pellet made of a swellable material is coated in a slow-degrading adhesive material, and this is placed in the socket. In this design, the adhesive coating helps keep the embodiment together while the drug payload is delivered, and then as the coating degraded/dissolves over time, water would diffuse through accessing the swellable material cause swelling of the material to 10× size which causes enough force to push out the rod.

The rate of swelling can be slowed down or sped up by changing the rate that water diffuses in the socket by modifying the silicone hinge. This can include, for example, a) changing length of the hinge and its thickness, b) adding slits on the side, c) including holes of varying diameter (e.g., 0.1 mm to 1 mm) on the caps of the hinge, d) increasing the number of holes on the caps or any combination of the above.

As can be appreciated the force of expulsion (which separates the rods from the linker) can be altered/affected by various factors. Forces of expulsion include, for example, amount of swellable material, type of material, speed of water access to material. Speed of water access can be controlled by water aperture (holes, slits) and by water-barrier coatings on the swellable component. Forces of retention include, for example, friction between rod and cap. It can be controlled by diameter of rod, material of rod, diameter of cap, material of cap) and presence of an adhesive (including the type and amount).

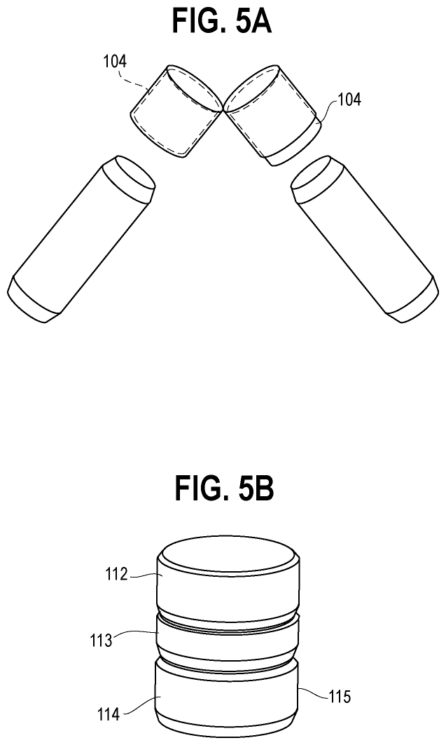

FIG. 5A depicts cylinders with linker complex (i.e., hinges) released. As noted above, rods or cylinders contain an API embedded in a polymer matrix. The cylinders are joined with a hinge to improve the size and consequently their retention in the bladder. The hinge portions are bound to at least a portion of the cylinder. The caps can be, for example, about 0.25 mm, about 0.5 mm, about 0.75 mm, about 1 mm, about 1.5 mm, about 2 mm or longer. As shown, they are 1.5 mm to 2.0 mm in length and contain a linker (i.e., pellet) of swellable material. As can be appreciated, the pellet should swell at a controlled rate to allow the drug product to remain inside the bladder for a retention period (e.g., one week, two weeks, three weeks, etc.).

FIG. 5B depicts a linker and its composition (i.e., internal swelling material). As shown, the linker includes three distinct layers (2-octyl-CA, PVP, PAA pellet+3×2-octyl-CA). The linker can include material that swells in solution such as, for example, poly(methyl methacrylate), poly(acrylic acid), Carbopol and hydroxypropyl methyl cellulose. In aspects, internal swelling material is about 0.25 mm in thickness. As can be appreciated, materials such as these typically swell to about 10× their original size.

As noted above, the drug particles should remain intact during a retention phase (e.g., one to two weeks). In aspects, the rate of swelling can be controlled via type of hydrophilic swellable polymers, their molecule weight (MW), salt or acetic acid blend, blend of hydrophilic swellable polymers with non-swellable hydrophobic materials and/or use enteric coatings.

Further, the drug products disassemble into separate parts for excretion in the urine. The disassembly function can be isolated to a single component of the implant that does not interfere with other functionalities. This approach allows different sections of the implant to perform distinct roles, enabling independent tuning of each component with minimal impact on the overall performance. For example, the implant can include rods made from a base material optimized for API diffusion, connectors primarily composed of flexible, non-degrading silicone, and a third component designed to ensure the connectors between the rods and the hinges fail at a predetermined rate or time. This third component, the linker, can be crucial as it connects different parts of the implant and eventually triggers its disassembly. When the implant's pieces are intact, its size or shape prevents natural expulsion during urination. Alternatively, the connector between the two hinges can be replaced from silicone to an erodible, elastic material which after erosion causes disassembly of socket with rod that can be voided.

### 4RC Drug Product

In certain embodiments, the intravesical drug product comprises a multi-segment chain architecture in which individual modules detach sequentially according to a predetermined timing profile. Modules can have dimensions selected from approximately 4×6 mm, 4×10 mm, 4×20 mm, or other geometries provided that post-detachment segments remain dimensioned for safe urethral elimination. The assembled chain can have an expanded profile up to approximately 24 mm or the effective diameter of an inflated Foley catheter balloon. Drug loading across modules can be uniform, tapered, stratified, or gradient-based, and release kinetics may be linked to module position within the chain.

FIG. 7 depicts an alternative design of a drug product. Rather than using a separate hinge, the cylinders are combined to form a chain-like structure with a bend in the center. In the modular multi-segment chain, each rod segment detaches in a predictable, sequence-dependent manner. Notably, the size/dimensions of the cylinders can be varied. For example, individual cylinders can be 4×20 mm, 4×10 mm, or smaller, provided the overall device profile remains about 24 mm.

As shown in FIG. 7, a unidirectional hinge (e.g., PCL-GA blend) can form the center of the linked cylinders. In aspects, the hinge is bendable only in one direction (e.g., up to about 180°) so that the products are amenable to catheter insertion. Moreover, the hinge can resist reverse bending to prevent premature expulsion. As with the RHL design, the particles undergo time-controlled degradation, enabling planned sequential detachment each component.

In embodiments, the hinge serves a dual function: (a) providing mechanical articulation and retention geometry during the retention phase, and (b) degrading or weakening to permit staged disassembly and excretion. The hinge can be composed of biodegradable elastomeric materials. For example, caprolactone copolymers with glycolide and/or lactide, polycaprolactone (PCL), PLGA, or blends thereof. Asymmetric hinge geometry may include thickness gradients, density gradients, or stress concentration features that enable controlled sequential detachment.

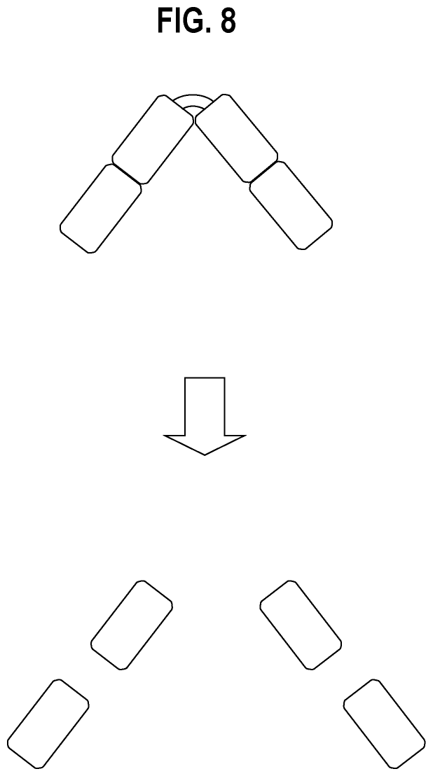

As shown in FIG. 8, The drug product can have a preferred conformation (i.e., angle) that it assumes post-insertion. This angle can be, for example, about 60° about 70°, about 80° or about 90°. A dissolvable polymer can be used as a linker to hold the cylinders in this position. The composition of the linker can be various materials known in the art and chosen based on, for example, dissolvability, buoyancy, stability, tear/tensile strength, moldability, elasticity, flexibility, moldability and hardness.

As with the RHL product, when separated from one another (i.e., after degradation/swelling), the individual components (i.e., cylinders and linker) can be excreted from the bladder. The composition of the degradable portion of the drug products can be selected based on a desired rate (i.e., retention period), such that e.g., at one week, about 25% of the product is cleared, at two weeks about 50% is cleared, etc.

As illustrated by the 4RC design in which a polymer coating functions as the hinge element, the hinge elements described herein are not limited to discrete, mechanically formed hinge bodies. In further embodiments, adjacent modules may be joined by alternative connection elements (also referred to herein as hinge elements) including, but not limited to: a dissolvable or degradable adhesive bond applied at the interface between adjacent modules; a degradable polymeric wrap, sleeve, or band encircling two or more adjacent modules so as to hold them in a connected configuration; a continuous coating applied over the junction between adjacent modules that degrades, erodes, or dissolves over time to permit separation; a degradable or non-degradable filament, suture, or thread passing through or around adjacent modules; and a mechanically interlocking joint or clip configured to release under predetermined conditions, such as mechanical fatigue from cyclic bladder forces. In each case, the connection element maintains the modules in a connected retention configuration during a therapeutic retention phase, and thereafter permits separation of the modules into individual components that are small enough for passive excretion. The rate of separation can be controlled by the material composition, thickness, molecular weight, cross-link density, or geometry of the connection element. In aspects, the connection element comprises a non-biodegradable material (e.g., silicone, polyurethane, or a non-degradable thermoplastic elastomer) that is configured to undergo mechanical failure (e.g., fatigue failure, creep rupture, or stress-induced fracture) under physiological conditions within the bladder over a predetermined time period, rather than chemical degradation. In aspects, the connection element comprises a biodegradable material that undergoes hydrolytic, enzymatic, or oxidative degradation. In aspects, the connection element combines biodegradable and non-biodegradable components.

### Non-Biodegradable Connection Elements and Mechanical Failure-Based Disassembly

In embodiments, the hinge element or connection element joining adjacent modules comprises a non-biodegradable material. As used herein, “non-biodegradable” refers to a material that does not undergo substantial chemical degradation (e.g., hydrolysis, enzymatic cleavage, or oxidative breakdown) under physiological conditions within the bladder over the intended retention period. Non-limiting examples of non-biodegradable materials suitable for use as connection elements include silicone (e.g., medical-grade polydimethylsiloxane), polyurethane, polyethylene, polypropylene, non-degradable thermoplastic elastomers, polyetheretherketone (PEEK), polytetrafluoroethylene (PTFE), and copolymers or blends thereof.

In such embodiments, the non-biodegradable connection element is configured to undergo controlled weakening through mechanical failure rather than chemical degradation. The bladder environment subjects retained devices to cyclic mechanical stresses, including compressive forces during bladder contraction, hydrodynamic shear forces during voiding, and impact and friction forces as the device contacts the bladder wall during normal patient movement. Over time, these cyclic stresses can induce progressive mechanical weakening of the connection element through one or more failure modes including fatigue failure (progressive structural damage from repeated cyclic loading below the material's ultimate tensile strength), creep rupture (time-dependent deformation and eventual fracture under sustained or repeated loading at physiological temperature), stress-induced fracture at designed stress concentration points (e.g., notches, reduced cross-sections, or geometric discontinuities intentionally incorporated into the connection element), and wear or abrasion at the interface between the connection element and the modules. The connection element can be dimensioned and geometrically configured so that mechanical failure occurs within a predetermined time window (e.g., from about 5 days to about 90 days after deployment) corresponding to the desired retention period.

In aspects, the non-biodegradable connection element includes one or more intentional stress concentration features to control the location and timing of mechanical failure. For example, the connection element may include a reduced cross-sectional area, a notch, a groove, a perforated region, a thinned wall, or a geometric discontinuity at a predetermined failure point. These features concentrate cyclic stress at the intended failure site, causing the connection to preferentially fracture at that location after a controlled number of loading cycles. The time to failure can be tuned by adjusting the material composition, the cross-sectional dimensions at the stress concentration feature, the wall thickness, and the overall geometry of the connection element. For example, a thinner silicone bridge between two modules will fatigue and fracture earlier than a thicker bridge of the same material, all else being equal. Similarly, selecting a material with a lower fatigue endurance limit (e.g., a stiffer thermoplastic elastomer rather than a highly elastic silicone) will produce an earlier mechanical failure for a given geometry.

In further aspects, the connection element may comprise a combination of biodegradable and non-biodegradable components. For example, a non-biodegradable structural element (e.g., a silicone bridge) may be combined with a biodegradable weakening element (e.g., a PCL-GA sheath or adhesive layer) such that degradation of the biodegradable component progressively reduces the load-bearing capacity of the non-biodegradable component until mechanical failure occurs. This hybrid approach can provide more predictable failure timing than either purely degradable or purely mechanical approaches alone. In aspects, the non-biodegradable connection element is configured to be small enough (e.g., less than about 6 mm in its largest dimension, and preferably less than about 4 mm) that, after separation from the modules, it can be passively excreted through the urethra during normal voiding without causing obstruction or requiring medical intervention. As used herein, the term “controlled weakening” encompasses both chemical degradation and mechanical weakening (including fatigue, creep, wear, and stress-induced fracture), such that a connection element that undergoes controlled mechanical weakening is understood to undergo “controlled weakening” within the meaning of the claims.

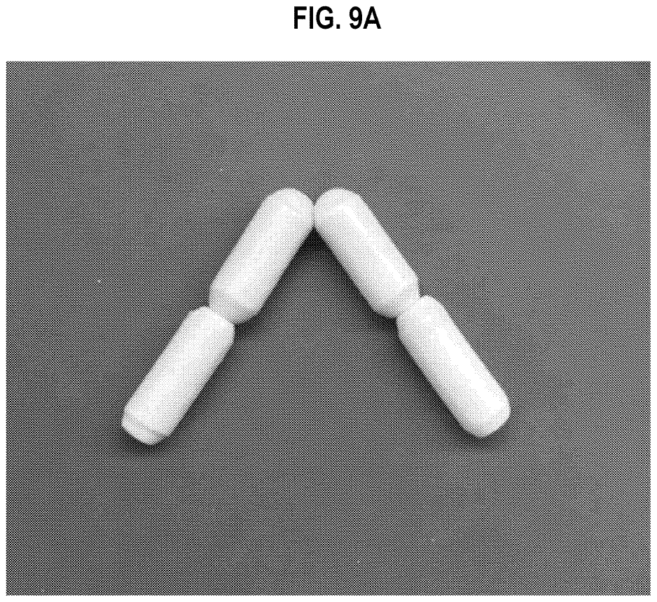

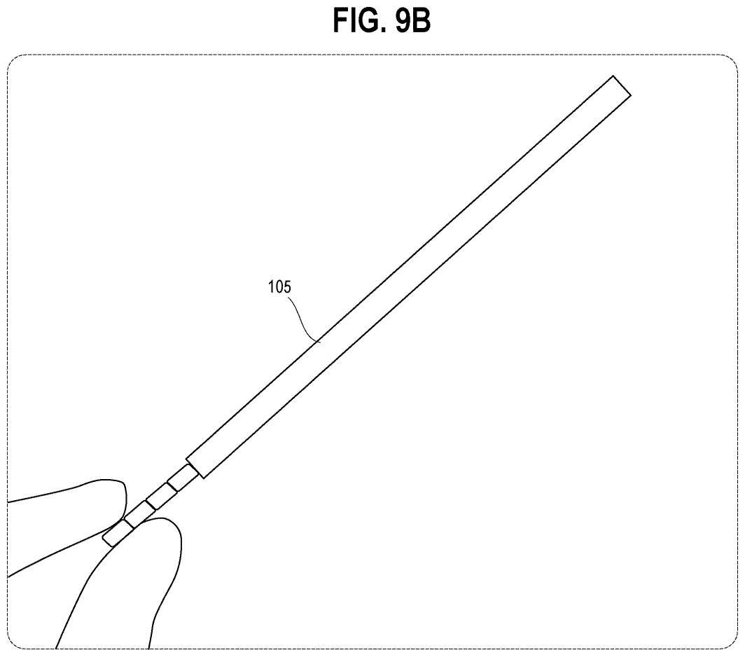

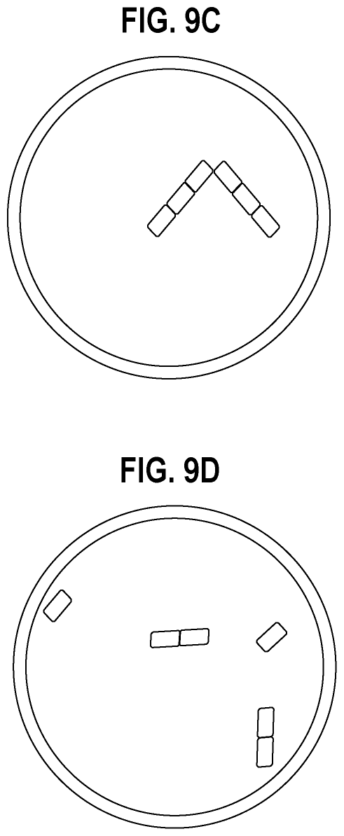

FIG. 9A-9D are images of the 4RC product showing the steps of implantation using a catheter (9B), drug delivery (9C) and product disassembly (9D). The drug product can be straightened into a configuration that is conducive to insertion. FIG. 2A depicts insertion into a catheter for administration into the bladder. As shown, the particles are aligned with one another and inserted through a catheter with a sheath. Thereafter, the particles revert to staggered arrangement (i.e., at an angle to each other in the retention environment) as shown in FIG. 9C. An active agent (i.e., payload) is released during this retention phase upon exposure to liquid (e.g., urine). During this phase, the components gradually dissociate from one another. As shown in FIG. 9D, the individual components are sufficiently small so that they are excreted during urination. Throughout the process, the product (and components) remain buoyant.

### Method of Manufacturing a 4RC Embodiment

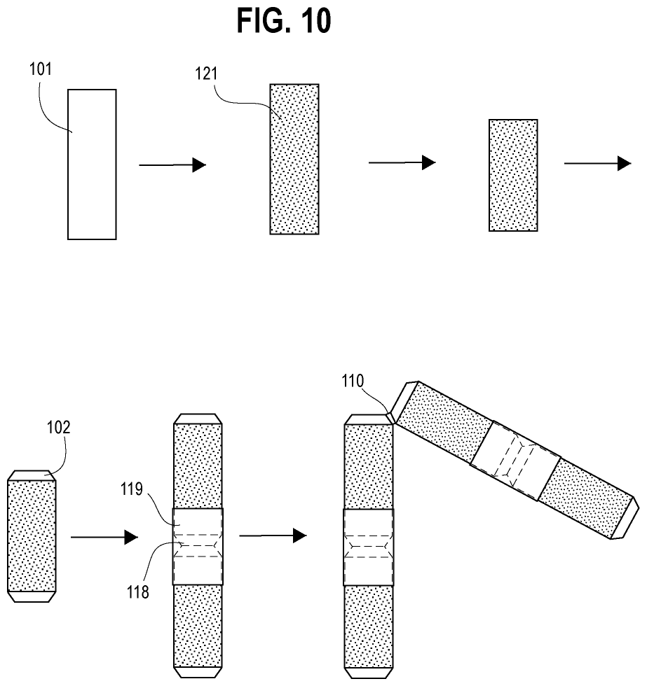

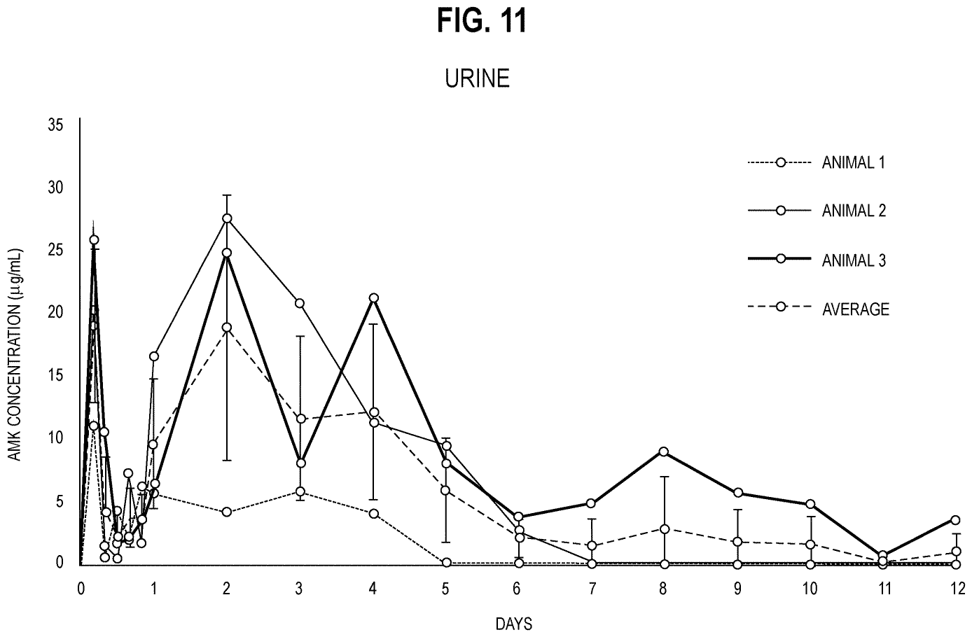

FIG. 10 is a schematic of the phases during the construction of the 4RC product. The process can be divided into six stages:
a) Forming and cutting,
b) EVA coating,
c) Cutting,
d) Rounding,
e) Joining cylinders with PCL-GA, and
f) Forming the product with a PCL-GA hinge.

Embodiments also include a method of manufacturing a modular intravesical drug-delivery device. The method can include steps of:
a) vertically extruding a polymer-drug melt via hot-melt extrusion to produce uniform rods;
b) cutting the rods into modules of variable length;
c) applying a coating via dip-coating, spray-coating, wrapping a pre-formed polymer sheet or film, co-extrusion, placement into a pre-formed polymer sleeve, or solvent-based coating;
d) attaching modules to hinge elements using heat-setting, solvent welding, or mechanical fixation; and
e) ensuring the assembled device maintains drug-uniformity and hinge integrity.

While vertical hot-melt extrusion is a preferred manufacturing method, it is understood that the rod-shaped modules may alternatively be formed by any suitable process, including but not limited to injection molding, compression molding, direct compression, additive manufacturing (3D printing), solvent casting, or combinations thereof. The selection of manufacturing process may be guided by the specific polymer-drug combination, desired drug uniformity, release rate, production scale, and target rod dimensions.

The following describes a representative method for manufacturing a four-rod-chain (4RC) intravesical drug product as depicted in FIG. 10. It is understood that variations in the specific materials, process parameters, and assembly sequences described herein are within the scope of the invention and may be adapted by one of ordinary skill in the art without departing from the principles disclosed.

### Step 1—Rod Formation

Rod-shaped modules are formed from a composition comprising an excipient and one or more active pharmaceutical ingredients. Each rod has an initial length of approximately 10 mm to 15 mm and a diameter of approximately 3 mm to 5 mm. Rods are inspected for dimensional conformity and surface integrity, and non-conforming rods are rejected.

### Step 2—Casing Application

Each rod is encased in a polymer layer that serves three principal functions: (1) controlling the rate and duration of active agent release from the rod over time, thereby lengthening the release profile relative to an uncoated rod; (2) protecting the rod against mechanical breakage during retention in the bladder; and (3) providing structural reinforcement that preserves rod geometry in the face of physiological forces. In a preferred embodiment, the casing comprises a non-biodegradable polymer such as ethylene-vinyl acetate (EVA), which does not degrade during the retention period and thereby maintains these protective and release-modulating functions for the duration of therapy.

The EVA casing can be formed by dissolving EVA in a suitable organic solvent at a concentration of about 3% to about 10% w/v. The rod is submerged in the solution, withdrawn, and allowed to dry. One or more additional coating cycles may be applied to achieve a target casing thickness and uniformity. The vinyl acetate content of the EVA, the polymer concentration in solution, the number of coating cycles, and the drying conditions may each be independently varied to tune the casing thickness, porosity, and drug release kinetics. In other embodiments, the casing may be applied by spray-coating, co-extrusion, wrapping a pre-formed polymer sheet or film around the rod, or placing the rod into a pre-formed polymer sleeve or tube. The casing material is not limited to EVA and may comprise any suitable polymer or polymer blend, including but not limited to PCL-GA, PCL, PLGA, polyurethane, or combinations thereof.

In an alternative embodiment, the polymeric casing itself serves as the drug-delivery matrix. In this configuration, the casing comprises at least one active agent dispersed or dissolved within the coating polymer. As the casing degrades in the bladder environment, the active agent is released in a controlled manner over the retention period. The rod core in this embodiment may be substantially free of active agent and serves primarily as a structural scaffold and buoyancy element, maintaining the device geometry and density required for bladder retention. This approach enables drug formulation independently of the rod matrix composition, permits the rod core to be optimized solely for mechanical and buoyancy properties, and allows drug loading to be controlled by adjusting the coating polymer composition, concentration of active agent in the coating solution, number of coating cycles, and casing thickness. The drug-loaded casing may comprise any of the coating polymers described herein, including biodegradable polymers such as poly(caprolactone-co-glycolide) (PCL-GA), polycaprolactone (PCL), poly(lactide-co-glycolide) (PLGA), or blends thereof. In some embodiments, the casing may comprise a first layer containing a first active agent and a second layer containing a second active agent or a different concentration of the first active agent, thereby enabling multi-drug or staged-release profiles from a single module. This embodiment is particularly advantageous where the active agent is sensitive to the thermal or mechanical conditions of rod formation, as the drug is introduced only during the coating step rather than during extrusion, molding, or compression of the rod core.

### Step 3—Trimming and End-Shaping

Each cased rod is trimmed to a final target length. Bare rod material extending beyond the casing is removed, and one or both rod ends are thermally shaped by contacting a heated surface at a temperature between about 50° C. and about 120° C. The rod is held against the heated surface briefly and rotated to produce a rounded end profile. This rounding step eliminates sharp edges for patient comfort and increases the contact surface area for subsequent joining. In some embodiments, a concave feature (dimple) is formed in one or both rod ends to further increase joint contact area, improve bond strength, and promote more predictable joint degradation behavior.

### Step 4—Knee Formation (In-Line Joining)

Pairs of finished rods are joined end-to-end to form collinear limbs using a biodegradable polymer adhesive. In a preferred embodiment, the adhesive comprises a solution of poly(caprolactone-co-glycolide) (PCL-GA) dissolved in a suitable organic solvent at a concentration of about 10% to about 25% w/v. The caprolactone-to-glycolide ratio may range from about 10:90 to about 90:10 and is selected to achieve a target balance of joint flexibility, bond strength, and degradation rate. Rods are loaded into alignment fixtures, their ends are dipped into the PCL-GA solution to a controlled depth, excess polymer is removed, and the rod ends are brought into contact and held in alignment until the joint has set. These in-line joints are referred to as “knees” and provide a rigid, biodegradable connection between adjacent rods within a limb. Where rod ends include dimples, the adhesive may additionally be applied into the dimple cavity prior to joining to increase bond area and improve joint reliability. In other embodiments, the knee joint may be formed by placing abutting rod ends into a pre-formed PCL-GA sleeve or tube, by wrapping a polymer film around the junction, by spray-coating the adhesive polymer onto the rod ends, or by co-extrusion of the adhesive polymer over the junction region.

### Step 5—Hip Formation (Angled Joining/Hinge Creation)

Two collinear limbs (each comprising two joined rods) are joined at an angle to form the hinge of the 4RC device. The limbs are loaded into a joining fixture that positions them at a predetermined resting angle of less than about 90°. The junction region is dipped into the same or a different PCL-GA solution as used for knee formation. Excess polymer is removed after each dip to control joint geometry. Multiple dip-and-cure cycles (e.g., 2 to 4 cycles) are applied to build the hip joint to a target thickness and strength. The resulting hip joint is flexible, enabling the 4RC device to assume a substantially collinear insertion configuration for passage through an appropriately sized catheter, and to return to a bent retention configuration once deployed in the bladder. The hip joint may be designed to degrade at a different rate than the knee joints, enabling sequential or staged detachment of individual modules. In other embodiments, the hip joint may be formed by placing the limb ends into a pre-formed PCL-GA sleeve or cuff, by wrapping a polymer film around the junction, by spray-coating the adhesive polymer onto the junction region, or by co-extrusion of the adhesive polymer over the angled junction. Over a predetermined period, the biodegradable polymer of both the knee and hip joints undergoes hydrolytic degradation in the presence of urine, leading to controlled weakening and eventual detachment of individual rod modules for passive urinary excretion.

Suitable rod matrix materials include hydrophobic waxes such as glyceryl tripalmitate (GTP), glyceryl tristearate, glycerol fatty acids, PCL, PLGA, EVA, polyurethane, polyacrylates, or blends thereof. Coating materials may include EVA, PCL-GA, PLGA, polyurethane, multilayer polymer coatings, or blends, with thicknesses ranging from approximately 10-250 μm. In certain embodiments, only the hinge component is biodegradable, while rod bodies and coatings may be non-degradable or diffusion-controlled.

As noted, the drug products (and components) are buoyant in urine. Buoyancy can be achieved through density modification, hollow geometry, trapped air inclusions, or low-density polymers. Alternatively, devices may be non-buoyant but geometrically configured to avoid obstruction.

### Agent Loading and Agent Combinations

As can be appreciated, the polymer composition and active agent can be chosen for a desired treatment regime. This allows:
Drug distribution across modules (uniform, tapered, or stratified),
Gradients linked to position within the chain, and
Release programming tied to the detachment order—this enables geometry-linked pharmacokinetic control.

In aspects, the polymeric coating is varied (i.e., “tuned”) to enable a desired release profile. Coating strategies (e.g., EVA, PCL-GA, or multilayer coatings) allow the same device architecture to support multiple release modes:
a) Burst release from selected modules,
b) Pulsatile/staggered release where different modules release at different timepoints, and
c) Multi-drug release, where each module contains a different drug, differential thickness or porosity across modules
   This enables programmable burst, sustained, staggered, or multi-drug release within one embodiment.

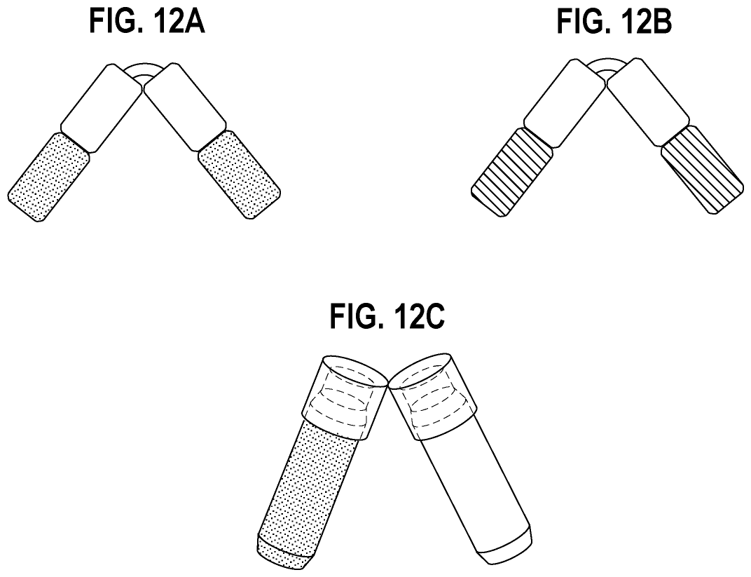

FIG. 12A-12C depict drug particles with different combinations of cylinders that form a drug product. FIG. 12A depicts a homogenous configuration in which each cylinder has the same polymer composition and active agent. FIG. 12B depicts a configuration in which cylinders have a different polymer composition. This allows administration of a “burst” or induction phase of an agent followed by a maintenance phase.

### Tunable Pharmacokinetic Profiles Via Adjustable Release Parameters

The drug release profile of the drug products described herein can be adjusted (“tuned”) through several complementary strategies, enabling the practitioner to select a desired pharmacokinetic output for a given therapeutic indication. Three primary mechanisms of release-profile tuning are described below: (i) variation of polymer coating composition, concentration, or thickness applied to individual rods; (ii) differential configuration of rods within a single drug product embodiment; and (iii) variation in the composition of the population of drug products administered into the bladder.

### (i) Variation of Polymer Coating Composition, Concentration, or Thickness

The rate and duration of drug release from each rod can be adjusted by varying the composition, solution concentration, or thickness of the polymer coating applied to the cylindrical rod. In aspects, the polymer coating comprises polycaprolactone-co-glycolide (PCL-GA), ethylene-vinyl acetate (EVA), or combinations thereof. A thicker coating, or a coating applied from a higher-concentration polymer solution, delays the onset of drug permeation through the coating membrane and extends the period of sustained release. Conversely, a thinner coating or lower-concentration coating shortens the lag time and produces a more rapid release profile. The thickness of the coating can be modulated by varying the number of dip-coating cycles, the polymer solution concentration, or the withdrawal speed during the dip-coating process, enabling tunable coating thicknesses in the range of approximately 10 to 250 μm. Similarly, the chemical composition of the coating (e.g., the ratio of caprolactone to glycolide in PCL-GA, or the vinyl acetate content of EVA) can be varied to alter the hydrophilicity, permeability, and erosion rate of the coating, and thereby the rate of drug release. By independently selecting the coating material and thickness for each rod within a drug product, the practitioner can engineer a desired release profile—including immediate release, delayed release, sustained release, or any combination thereof—from a single drug product.

(ii) Differential Rod Configuration within a Single Drug Product Embodiment

In embodiments, the individual rods that constitute a single drug product need not be identical. In aspects, one or more rods within a single drug product may be uncoated (i.e., lacking a polymer coating), while the remaining rods are provided with a polymer coating. An uncoated rod releases its drug load rapidly as a burst dose upon exposure to urine, while the coated rods release drug more slowly over an extended period. This configuration produces a biphasic pharmacokinetic profile from a single administered drug product: an initial burst or induction phase derived from the uncoated rod(s), followed by a sustained maintenance phase derived from the coated rod(s).

In aspects, a drug product comprises four rods, wherein one rod is uncoated and three rods are polymer-coated. The uncoated rod provides a burst release of drug over a short initial period (e.g., approximately one to seven days), while the polymer-coated rods provide sustained release over a longer period (e.g., approximately 30 to 90 days). In aspects, all four rods contain the same active agent. For example, a drug product in which one uncoated rod delivers a burst release of an antibiotic (e.g., amikacin, nitrofurantoin, or fosfomycin) can rapidly achieve bactericidal concentrations in the bladder to treat an acute urinary tract infection, while the three remaining coated rods provide sustained low-level release of the same antibiotic for ongoing prophylaxis against recurrent infection.

In further aspects, the rods within a single drug product may contain different active agents, enabling simultaneous dual-drug delivery with independently programmable release kinetics for each agent. For example, one or more rods within a drug product may be loaded with a first active agent and configured for burst release (e.g., uncoated or lightly coated), while the remaining rods are loaded with a second active agent and configured for sustained release (e.g., coated with a thicker or less permeable polymer layer). As a specific non-limiting example, a drug product comprising one uncoated rod loaded with an antibiotic can deliver a burst of antibiotic to clear an active urinary tract infection, while three coated rods loaded with an antispasmodic agent (e.g., oxybutynin) provide sustained release of the antispasmodic to address concurrent overactive bladder symptoms over an extended period. This combination addresses two clinically common, co-occurring urological conditions—acute infection and bladder overactivity—from a single intravesical drug product.

(iii) Variation in the Population of Drug Products Administered into the Bladder

In embodiments, a plurality of drug products administered together into the bladder as a single treatment session need not be identical to one another. Rather, the population of drug products placed into the bladder may be heterogeneous in composition, drug loading, or coating, such that different subsets of the administered drug products release different active agents or release the same agent at different rates.

For example, in an administered set of four drug products, one drug product may release a first active agent (e.g., an antibiotic) while the remaining three drug products release a second active agent (e.g., an antispasmodic agent such as oxybutynin, or a chemotherapeutic agent). This population-level approach allows flexible combination therapy to be assembled from individually manufactured, single-agent drug products, without requiring each drug product to incorporate multiple active agents or heterogeneous rod compositions. The ratio of drug product types within the administered population can be adjusted to alter the relative doses of each agent delivered to the bladder. In aspects, the population comprises drug products that differ in active agent identity, active agent loading, polymer matrix composition, polymer coating material, coating thickness, or any combination thereof, such that the overall pharmacokinetic profile delivered to the bladder is a composite of the individual release profiles of each drug product type in the population.

### Alpha Shell/Beta Core

Drug-loaded rods used in implants are prone to breaking due to low fracture strength, which can impact long-term structural integrity and patient safety. Embodiments of the invention include a dual-phase polymorphic structure. In aspects, an alpha shell (that is typically stronger) surrounds a beta core (for buoyancy). Applicants have discovered that this design enhances fracture strength while maintaining buoyancy in urine/water.

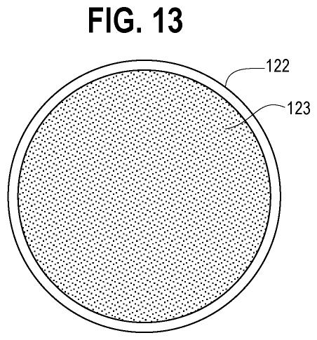

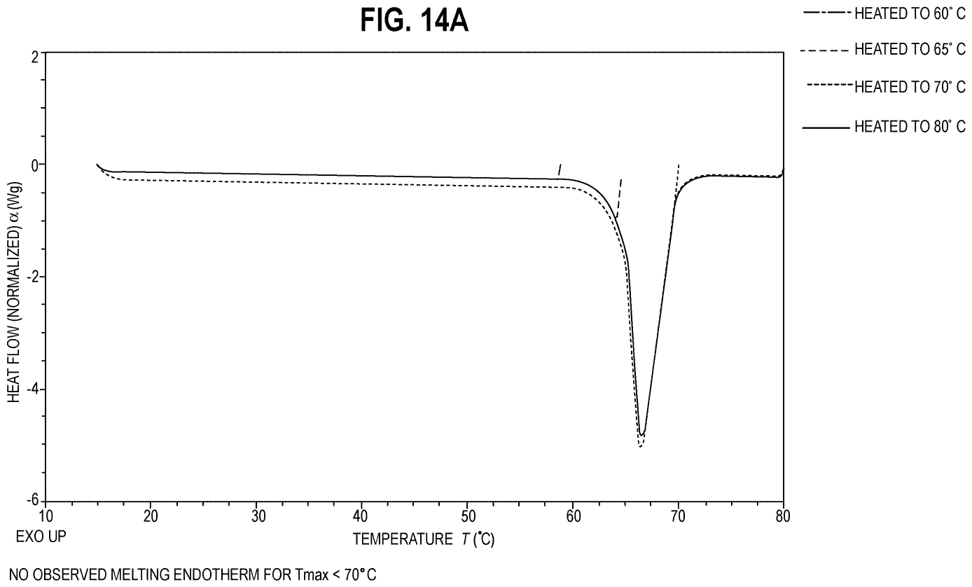

FIG. 13A is an image of a beta-phase crystalline core for controlled drug release and stability. FIG. 13B is an image of an alpha-phase crystalline shell that can provide enhanced mechanical strength and elasticity. FIG. 13C is an image that shows the beta polymorph core and the alpha polymorph shell.

In addition to optimizing the ASBC (Alpha Shell Beta Core) geometry, Applicants have discovered that the choice of carrier material significantly influences mechanical performance and phase stability. Specifically, polyglycerol esters of fatty acids (PGFAs), such as Witepsol® PMF 222 or 282, can be employed as alternative or complementary matrix materials. These fully esterified PGFAs are characterized by higher melting points (59.4° C.-72.5° C.), low hydrophilic-lipophilic balance (HLB), and absence of polymorphic transition under physiological conditions. Unlike lipid-based carriers that exhibit polymorphic phase shifts (e.g., from β to a or vice versa), Witepsol® PMF materials retain a consistent solid-state structure, reducing the risk of mechanical degradation over time. Furthermore, their miscibility and tunable wettability enable precise control over drug release kinetics and structural homogeneity, contributing to enhanced fracture strength and performance reliability of the implant.

### Dip Coating for Improving Integrity of Drug-Delivery Rods

Drug-delivery rods used in implants can be prone to mechanical breakage due to brittleness, which compromises drug release, retention, structural integrity, and patient safety. Additionally, controlling degradation rate is essential for ensuring a sustained therapeutic effect.

Embodiments include a protective dip-coating process using polycaprolactone (PCL), polycaprolactone-lactide (PCL-LA), and polycaprolactone-glycolide (PCL-GA), EVA copolymers, butyl-methacrylate and copolymers or other type of elastomers that:
Improve mechanical durability by forming a flexible, impact-resistant shell.
Improve integrity during handling, insertion, and in vivo retention.
Allow preserved length of rod in the presence of fractured segments
Control degradation kinetics, drug release kinetics based on polymer composition, molecular weight (MW), and intrinsic viscosity (IV).
Provide tunable coating thickness (10-250 μm) using single or multiple dip-coating cycles.

### Methods of Use

In embodiments, the particles within the pharmaceutical formulation include (a) a pharmacologically active agent and (b) a controlled release carrier. By gradual reduction in mass and/or other means, the particles release the active agent into the bladder during an extended drug delivery time period. The components of the particles are described further herein.

As can be appreciated, the selection of pharmacologically active agent is dependent on the indication. Active agents that are administrable via intravesicular administration of a particle formulation of the invention are generally, although not necessarily, selected from the following categories: anti-infective agents, including anti-bacterial, anti-fungal, and anti-viral agents; chemotherapeutic agents; anti-inflammatory agents; anesthetic agents; analgesic agents; diuretic agents; coagulants and anti-coagulants; biologics; agents for treatment of incontinence, including overactive bladder (including antimuscarinic agents, b3-adrenergic receptor agonists, anesthetic agents, and analgesic agents); renin-angiotensin-aldosterone system (RAAS) inhibitors; agents for modulating an immune response, agents for treating kidney stones; agents for gene therapy and contrast agents for diagnostics and monitoring. In preferred embodiments, the active agent is selected for local intravesical therapy, including antibiotics, chemotherapeutics, anesthetics, anti-inflammatory agents, immunotherapeutics, or agents for bladder-specific disorders.

As described above, drug formulations are most conveniently administered to a patient in a compacted form. For insertion, the product can have an effective diameter of, for example, about 5.5 mm. Alternatively, the product can have a diameter of about 3 mm, about 4 mm, about 5 mm, about 6 mm, about 7 mm or about 8 mm. Within the bladder, the product can have an effective diameter of, for example, about 24 mm. Alternatively, the product can have a diameter of about 12 mm, about 14 mm, about 16 mm, about 18 mm, about 20 mm, about 22 mm, about 26 mm, about 28 mm or about 30 mm.

As described herein, the capsules can expand or conform to a larger size within the bladder to resist passage into the urethra over a desired residence period. In aspects, the particles have a combined volume of about 60 mm³ to about 1,000 mm³. In aspects, the particles have a combined volume of about 60 mm³, about 125 mm³, about 150 mm³, about 175 mm³, about 200 mm³, about 250 mm³, about 300 mm³, about 350 mm³, about 400 mm³, about 450 mm³, about 500 mm³, about 600 mm³, about 700 mm³, about 800 mm³, about 900 mm³, about 1,000 mm³, or larger. In aspects, one of the cylinders is released from the hinge for excretion. Alternatively, both cylinders can be released from the hinge.

In embodiments, disassembly of the rod-hinge-linker complex occurs in about 5 days, about 7 days, about 10 days, about two weeks, about three weeks, about 1 month, about 45 days, about 60 days, about 75 days, about 90 days or longer.

In yet further aspects, the drug products are connected by a biodegradable hinge, the biodegradable hinge designed to maintain its integrity (i.e., not dissolve) until after one voiding cycle, two voiding cycles, three voiding cycles, four voiding cycles, five voiding cycles, six voiding cycles, seven voiding cycles, eight voiding cycles, nine voiding cycles, 10 voiding cycles, 11 voiding cycles, 12 voiding cycles, 13 voiding cycles, 14 voiding cycles, 15 voiding cycles, 16 voiding cycles, 17 voiding cycles, 18 voiding cycles, 19 voiding cycles, 20 voiding cycles, 25 voiding cycles, 30 voiding cycles, 35 voiding cycles, 40 voiding cycles, 45 voiding cycles, 50 voiding cycles, 55 voiding cycles, 60 voiding cycles, 65 voiding cycles, 70 voiding cycles, 75 voiding cycles, 80 voiding cycles, 90 voiding cycles, 100 voiding cycles, 110 voiding cycles, 125 voiding cycles, 150 voiding cycles, 175 voiding cycles, 200 voiding cycles, 225 voiding cycles, 250 voiding cycles, 275 voiding cycles, 300 voiding cycles, 325 voiding cycles, 350 voiding cycles, 375 voiding cycles, 400 voiding cycles, 450 voiding cycles, 500 voiding cycles, 600 voiding cycles, 700 voiding cycles, 800 voiding cycles, 900 voiding cycles, 1000 voiding cycles or more.

In aspects, the two sides of the hinge are joined by a connector. The connector can allow a health care professional to physically remove the drug products from the bladder (while in the retention phase) if necessary. As can be appreciated, the connector should be of sufficient length so that it can be secured using standard forceps or alligator clamps. The connector can be, for example, about 0.5 mm in length, about 1 mm in length, about 1.5 mm in length, about 2 mm in length, about 3 mm in length, about 4 mm in length, about 5 mm in length, about 6 mm in length, about 7 mm in length, about 8 mm in length, about 9 mm in length or longer.

In aspects, administration of the drug products and devices of the present invention can improve urinary continence in a subject or patient by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99% or at least 99.5%.

In aspects, administration of the drug products and devices of the invention can reduce signs/symptoms of a disease of the bladder/urinary tract/kidney by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 97%, about 98%, about 99% or about 100%.

In aspects, administration of the drug products and devices of the present invention can reduce signs/symptoms of a disease of the bladder/urinary tract/kidney by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99% or at least 99.5%.

In aspects, intravesical administration of a drug product using devices/methods of the invention can reduce reported patient discomfort (as compared to using a conventional catheter) by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99% or at least 99.5%.

In aspects, administration of the drug products and devices of the invention decreases the time needed for intravesical administration of a drug product (e.g., including prep time) by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 97%, about 98% or about 99%, compared to conventional methods (e.g., hand loading a catheter). In aspects, the devices and methods allow intravesical administration with fewer trained staff (i.e., administration by a single health care professional/technician).

In embodiments, the devices/methods described herein can deliver drug particles wherein the mean diameter of the particles or drug delivery product is about 50 nanometers, about 100 nanometers, about 200 nanometers, about 350 nanometers, about 500 nanometers, about 1,000 nanometers, about 2,000 nanometers, about 3,000 nanometers, about 5,000 nanometers, about 10,000 nanometers, about 10 micrometers, about 20 micrometers, about 30 micrometers, about 50 micrometers, about 100 micrometers, about 150 micrometers, about 250 micrometers, about 500 micrometers, about 750 micrometers, about 1,000 micrometers, about 1,500 micrometers, about 2,000 micrometers, about 3,000 micrometers, about 5,000 micrometers, about 10,000 micrometers, about 1 millimeter, about 1.25 millimeters, about 1.35 millimeters, about 1.45 millimeters, about 1.5 millimeters, about 1.65 millimeters, about 1.75 millimeters, about 1.85 millimeters, about 2 millimeters, about 2.15 millimeters, about 2.25 millimeters, about 2.5 millimeters, or about 3 millimeters or greater.

In aspects, the controlled release particles described herein are cylinder-shaped (i.e., barrel-shaped or cylindrical) or substantially cylinder-shaped. In aspects, they have a volume of about 60 mm³ to about 1,000 mm³. In aspects, the particles have a combined volume of about 60 mm³, about 125 mm³, about 150 mm³, about 175 mm³, about 200 mm³, about 250 mm³, about 300 mm³, about 350 mm³, about 400 mm³, about 450 mm³, about 500 mm³, about 600 mm³, about 700 mm³, about 800 mm³, about 900 mm³, about 1,000 mm³, or larger. In aspects, one of the cylinders is released from the hinge for excretion. Alternatively, both cylinders can be released from the hinge.

### EXAMPLES

The following non-limiting examples are provided for illustrative purposes only in order to facilitate a more complete understanding of representative embodiments now contemplated. These examples are intended to be a mere subset of all possible contexts in which the components of the formulation may be combined. Thus, these examples should not be construed to limit any of the embodiments described in the present specification, including those pertaining to the type and amounts of components of the formulation and/or methods and uses thereof.

### Example 1

### Method of Producing Reinforced Drug-Delivery Rods

Drug-loaded rods used in implants can be prone to breaking due to low fracture strength, which can impact long-term structural integrity and patient safety. In this example, rods are produced with a dual-phase polymorphic structure—an alpha shell (stronger) surrounding a beta core (buoyant)—to enhance integrity/strength, and maintain buoyancy in urine/water.

The method includes the following steps:
a) GTP excipient is melted between 85-110° C. (optimally 105° C.);
b) A shell-cooling step (0-20° C. for 5-10 s, ideally 0-10° C. for 5 s) induces alpha-phase crystallization;
c) A core-curing step at 45-55° C. for ≥20 min promotes beta-phase formation;
d) Alpha shell thickness is increased by prolonging cooling time; and
e) Beta core diameter is increased by adjusting the curing temperature near 50° C.

Table 1 is a summary of processing parameters for alpha shell—beta core formation. Table 2 shows cooling rate and “ramp-up” parameters.

TABLE 1

| | Temperature | Ideal | |
|---|---|---|---|
| Step | Range (°C) | Conditions | Impact on Structure |
| Melting GTP | 85-110 | 105 | Ensures full excipient |
| | | | liquefaction |
| Shell Cooling | 0-20 for | 0-10 for | Forms alpha shell via |
| | 5-15 s | 8-12 s | rapid solidification |
| Core Curing | 45-55 for | 50 for | Induces beta-phase |
| | ≥20 min | 20 min | crystallization |

TABLE 2

| Factor | Effect on Phase Transition |
|---|---|
| Prolonged cooling | Thicker alpha shell (stronger, |
| | higher fracture strength) |
| Brief cooling | Thinner alpha shell (weaker |
| | fracture strength) |
| Higher curing | Increases beta core diameter |
| temp (~50 °C) | (improve buoyancy) |

Thereafter, a process for “tuning” mechanical properties can be used. The process can include:
a) Controlled cooling rates and ramp-up times dictate phase transformation kinetics;
b) Light transmission tests and differential scanning calorimetry (DSC) confirm alpha/beta polymorph presence; and
c) Twin-screw extrusion parameters, including screw speed and pressure, influence crystallization and phase separation.

The product is a reinforced drug-delivery rod. Each rod has a beta-phase crystalline core for controlled drug release and stability and an alpha-phase crystalline shell providing enhanced mechanical strength and elasticity.

Next, the strengths of the rods were studied. Mean break strength of alpha shell and beta core rods improved from beta rods only that are brittle and buoyancy was maintained as shown below in Table 3.

TABLE 3

| | Mean Break | Std Dev |
|---|---|---|
| | Strength (N) | (N) |
| Sterilized ASBC rods | 5.59 | 1.68 |
| Sterilized Beta Rods | 2.85 | 0.62 |

The product was further studied to ensure that buoyancy over extended periods (i.e., 100% floating for 14+ days in urine). The results are shown below in Table 4.

TABLE 4

| | 0 days | 5 days | 6 days | 14 days |
|---|---|---|---|---|
| Sterilized ASBC rods buoyant | 100% | 100% | 100% | 100% |
| in urine media | | | | |

The results indicated that the “alpha shell-beta core” structure provided mechanical reinforcement. Further, dual-phase crystalline engineering enhanced rod toughness while maintaining flexibility. The characterized GTP thermal properties are shown below in Table 5.

TABLE 5

| Polymorph | T_{crystallization} (°C) | Tₘₑₗₜ (°C) |
|---|---|---|
| α | 40 | 46 |
| β′ | n/a | 59.5 |
| β | 49 | 66 |

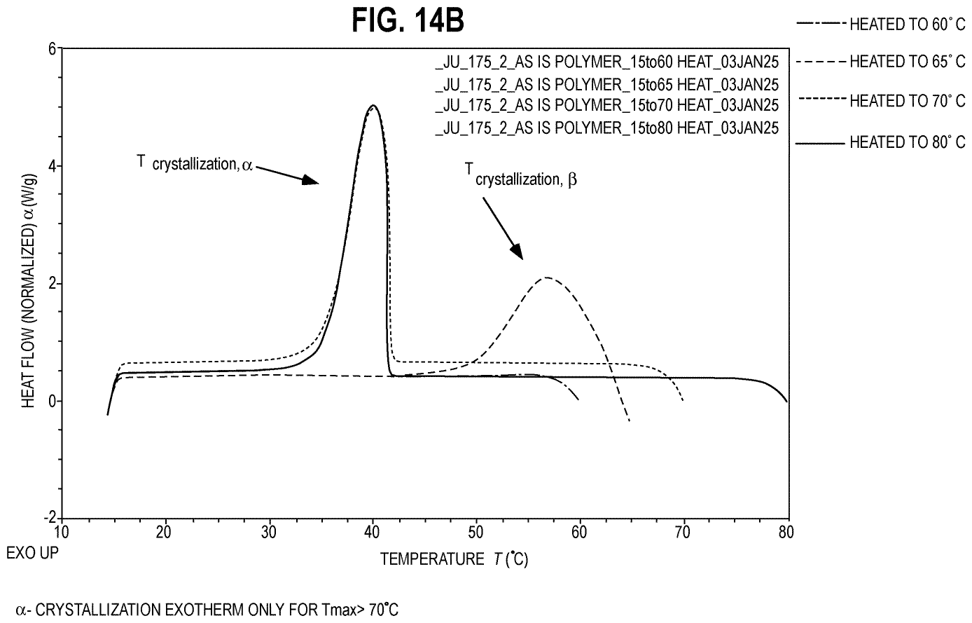

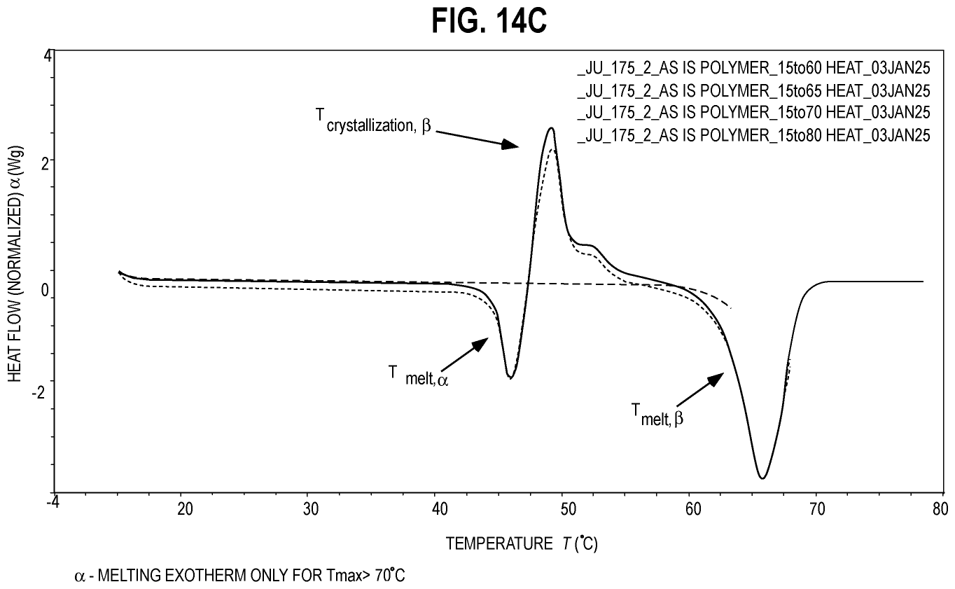

The rods were further studied to characterize isotropic melt (i.e., occurring in the same in all directions). FIG. 14A is a graph of temperature (C) versus normalized heat flow (W/g) that shows the minimum temperature required to generate isotropic melting. FIG. 14B is a graph that shows α-crystallization exotherm. FIG. 14C is a graph that shows α-melting endotherm.

The rods had a tunable alpha shell thickness (e.g., 0.1 mm to 2 mm) for customized mechanical and buoyancy properties. Further, the beta-core stabilization prevented uncontrolled swelling and premature drug release.

Thermal processing control can be used for phase selection. For example, precise cooling and curing temperature ramps control polymorphic conversion. Prolonged cooling leads to a thicker alpha shell; Prolonged curing leads to a denser beta core. Further, quick phase transitions via twin-screw extrusion can optimize consistency.

The products can be subject to analytical validation for polymorphic integrity. This can include:
Light test (translucency vs. opacity) visually confirms alpha vs. beta formation.
X-ray Crystallography: Confirms phase distribution
DSC analysis differentiates phase-specific melting points and heat flow signatures.

### Example 2

### Dip Coating Drug-Delivery Rods

Drug-delivery rods used in implants are prone to mechanical breakage due to brittleness, which compromises drug release, retention, structural integrity, and patient safety. Additionally, controlling degradation rate is essential for ensuring a sustained therapeutic effect. Provided herein is a protective dip-coating process using polycaprolactone (PCL), polycaprolactone-lactide (PCL-LA), and polycaprolactone-glycolide (PCL-GA), EVA copolymers or other type of elastomer that:
Improves mechanical durability by forming a flexible, impact-resistant shell;
Improves integrity during handling, insertion, and in vivo retention;
Allows preserved length of rod in the presence of fractured segments;
Controls degradation kinetics, drug release kinetics based on polymer composition, molecular weight (MW), and intrinsic viscosity (IV); and
Provides tunable coating thickness (e.g., 10-250 μm) using single or multiple dip-coating cycles.

In this example, the rods are produced by a method of dip-coating rods to improve mechanical strength. Each rod is immersed in a polymer solution for controlled coating deposition. The thickness of the coating can be adjusted by different steps, including:
Varying the number of dip-coating cycles (1× vs. multiple coatings);
Polymer molecular weight (MW), measured by intrinsic viscosity (IV), which determines solution viscosity and elasticity;
Caprolactone-to-glycolide ratio, controlling degradation rate and mechanical flexibility;
Concentration (w/v) of the polymer in solution.

In this example, the polymer properties were optimized for mechanical reinforcement. Applicants found that a 45:55 caprolactone-to-glycolide ratio provided an optimal balance of elasticity and degradation rate. Further, intrinsic viscosity (IV) of ~1.5 dL/mL ensured a highly viscous, flexible coating. A single dip-coating cycle with PCL-GA (45:55) resulted in a 250 μm thick layer around the rod.

Applicants also studied the role of intrinsic viscosity (IV) in coating strength and thickness and determined:
Intrinsic viscosity (IV) measures the polymer's molecular weight (MW) and viscosity in solution.
A higher IV (e.g., 1.5 dL/mL)→Higher MW→Higher viscosity→More elastic, stronger coating.
A lower IV (e.g., 0.5 dL/mL)→Lower MW→Lower viscosity→Thinner, more brittle coating.
   For example, a PCL-GA polymer with 1.5 dL/mL IV forms a highly elastic and impact-resistant coating, reducing rod fractures.

Applicants also studied the effect of dip coating speed and solution concentration and determined:
A higher glycolide content→Faster degradation.
A higher molecular weight→More elastic and slower degradation.
Multiple coatings→Increased structural integrity for extended retention.
Faster dipping speeds lead to more uniform and consistent coatings.
Slower dipping speeds cause surface tension effects, pulling solution away from the rod and resulting in thin or uneven coatings.
Time spent in solution had a significant impact on coating thickness at shorter immersion times (e.g., less than about 3 seconds), but had little additional impact beyond approximately 3 seconds of immersion.
A higher solution concentration (e.g., up to 30% w/v) results in thicker coatings.
Surface morphology of the rod (e.g., roughness, porosity, and geometry of features such as dimples) had a significant impact on coating thickness and mechanical strength, with rougher or more featured surfaces retaining thicker and more adherent coatings.
   Key processing parameters for dip coating formation are summarized below.

TABLE 6
Variable Controlling Coating Thickness and Elasticity

| Processing Parameter | Effect on Coating Properties |
|---|---|
| Caprolactone:Glycolide | Controls elasticity vs. degradation rate |
| Ratio (45:55) | |
| Intrinsic Viscosity (IV) | Ensures high solution viscosity & |
| of 1.5 dL/mL | mechanical resilience |
| Number of Dip Coatings | Adjusts thickness and structural durability |
| (1x vs. multiple layers) | |
| Dipping Speed | Faster speeds = uniform coating, slower |
| | speeds = uneven coating |
| Solution Concentration | Higher concentration = thicker coatings |
| (1%-30% w/v) | |
| Solvent Choice | Impacts polymer dissolution and coating |
| | uniformity |

TABLE 7
Solvent Selection for PCL & PCL-GA Processing

| Polymer | |
|---|---|
| Type | Compatible Solvents for Dip-Coating |
| PCL | Chloroform, Acetone, Tetrahydrofuran (THF), |
| | Dichloromethane (DCM) |
| PEG-PCL | Dimethylformamide (DMF), Dichloromethane |
| | (DCM), THF |
| PCL-GA | Chloroform, DCM, THF, Acetone |
| (45:55) | |

The rods can be analyzed via one or more of (a) optical and/or microscopic analysis, (b) mechanical testing and (c) biodegradation testing. Scanning Electron Microscopy (SEM) can be used to confirm coating uniformity and adhesion. Light microscopy can measure layer thickness and structural integrity.

Break strength testing can be performed before and after coating application. Elastic modulus testing can be used to compare coated and uncoated rods. Biodegradation testing can include measuring mass loss over time in simulated urine environment to correlate degradation kinetics with coating properties.

### Example 3

### Intravesical Particles to Treat UTI (RHL Construct)

In this example, a female patient visits a healthcare provider with signs/symptoms of a UTI. The provider notes that the patient has had three UTI's within a six-month period. Previously, she has been prescribed oral antibiotics (i.e., amoxicillin or nitrofurantoin). Although prophylactic oral antibiotics are an option, the patient wishes to avoid them. Further, the healthcare provider acknowledges the risk of antibiotic resistance.

The provider presents the option of using intravesical controlled release particles. Conventional drug delivery systems (e.g., tablets, capsules, etc.) can suffer from poor bioavailability and fluctuations in plasma drug levels. The use of intravesical particles allows drug delivery at a specified controlled rate and at a target site (i.e., the bladder) for greater efficacy and safety.

The intravesical particles are stacked and placed into a catheter as shown in FIG. 2A. The particles are administered through the urethra into the patient's bladder. The particles assume the retention configuration within the bladder such that they have a diameter of 10 mm or higher. Due to their size, the particles are resistant to being voided during urination. Amikacin is released from the cylinders into the bladder.

A portion of the cylinders (i.e., linker) includes a material that swells upon exposure to liquid as shown in FIG. 4. This occurs during a retention phase of about ten days. The swelling causes the cylinders to be released from the caps. Thereafter, the individual cylinders and cap portion are readily excreted in the urine (i.e., the elimination phase).

In this example, particles remain intact for about ten days. The concentration of amikacin in the bladder remains relatively constant during this period.

The patient reports alleviation of symptoms within 24 hours of the procedure. The patient has no discomfort nor any feeling of the presence of the particles. Approximately 48 hours after administration, she has no signs/symptoms of UTI and more than 90% of the cylinders remain in her bladder (as observed by ultrasound). The patient is evaluated one week later and has no signs/symptoms of infection. Further, the provider notes that there is no detectable circulating amikacin though it is present in the bladder at an effective concentration. Thereafter, the patient is advised to maintain routine visits to her healthcare provider and to report any signs/symptoms of possible UTI.

After ten days, the cylinders have separated from each other and are voided during urination. Within two weeks, cylinders are absent from the bladder (as observed by ultrasound). The patient is advised to contact the clinic if she experiences any later signs/symptoms of UTI. If needed, the particles can be “tuned” so to increase the duration of the retention period. The particles can be coated with a thicker polymeric coating to increase the retention period. The patient can receive a lower dose of amikacin for a longer retention period (e.g., 30 days).

### Example 4

### Intravesical Particles to Treat UTI and OAB (RHL Construct)

In this example, a female patient visits a healthcare provider with signs/symptoms of a UTI. She also complains of sudden, uncontrollable urge to urinate. The provider diagnoses the patient with UTI and overactive bladder (OAB). The provider presents the option of using intravesical controlled release particles. The particles are configured to release two agents: amikacin to treat an infection and oxybutynin to treat overactive bladder.

As described above (EX. 3) the intravesical particles are administered in a stacked arrangement through the urethra into the patient's bladder. The particles assume the retention configuration within the bladder. The particles release both active agents during a retention phase of about ten days. Swelling causes the cylinders to be released from the caps and individual components are excreted in the urine (i.e., the elimination phase).

In this example, particles remain intact for about ten days. The concentrations of amikacin and oxybutynin in the bladder remain relatively constant during this period.

The patient reports alleviation of symptoms within 72 hours of the procedure. She has no signs/symptoms of UTI nor overactive bladder. The patient is advised to maintain routine visits to her healthcare provider and to report any signs/symptoms of possible UTI.

### Example 5

Intravesical Particles to Treat IC (4RC construct)

Interstitial cystitis (IC), also known as bladder pain syndrome (BPS), is a chronic condition characterized by bladder pain, pressure, and discomfort that is not caused by a urinary tract infection (UTI). In this example, a female patient visits a healthcare professional complaining of lower abdominal pain and the urge to frequently urinate. She is diagnosed with interstitial cystitis (IC)/bladder pain syndrome (BPS). The physician recommends treatment with intravesical administrated anti-inflammatories.

In this example, the patient is administered “4RC” particles as depicted in FIG. 7. The particles are administered in a stacked arrangement through the urethra, as depicted in FIG. 2A. Thereafter, the cylinders revert to the second configuration (i.e., retention phase). During the retention phase, the cylinders release therapeutic amounts of GAG layer restoratives in combination with an anti-inflammatory agent (NSAID).

As shown in FIGS. 7 and 8, the cylinders gradually separate from one another upon exposure to liquid (i.e., urine). This occurs during the retention phase as a polymer coating gradually degrades in solution. Thereafter, the individual cylinders are readily excreted in the urine (i.e., the elimination phase).

In this example, particles remain intact for about 30 days. The concentration of active agents in the bladder remains relatively constant during this period.

The patient reports alleviation of symptoms within 48 hours of the procedure. One week after administration, she has no signs/symptoms of IC and more than 90% of the cylinders remain in her bladder (as observed by ultrasound). Thereafter, the patient is advised to maintain routine visits to her healthcare provider and to report any discomfort. After 30 days, the cylinders have separated from each other and are voided during urination. Within 45 days, cylinders are absent from the bladder (as observed by ultrasound).

The composition of the cylinders can be modified to extend the duration of the retention phase. The polymeric coating can be modified or “tuned” to extend the retention phase. One approach relates to the use of an enteric coating. The outer layer can reduce water movement/absorption. The coating gradually dissolves upon exposure to urine in the bladder.

### Example 6

### Intravesical Particles to Treat Muscle Invasive Bladder Cancer (MIBC) (4RC Construct)

In this example, a 75-year-old male patient visits a healthcare provider and complains of blood in the urine, pain with urination, and low back pain. The provider conducts a visual inspection of the bladder via cystoscopy. Pathological examination confirms the presence of tumor cells. The patient is diagnosed with muscle invasive bladder cancer (MIBC).

The provider recommends surgery in combination with intravesical chemotherapy. Surgery combined with adjuvant intravesical administration (i.e., direct administration of the chemotherapeutic agent into the bladder) of chemotherapeutic or immunotherapeutic agents to help prevent or delay the incidence and severity of recurrence.

As noted, conventional drug delivery systems (e.g., tablets, capsules, etc.) can suffer from poor bioavailability and fluctuations in plasma drug levels. The use of intravesical particles allows drug delivery at a specified controlled rate and at a target site for greater efficacy and safety.

The patient receives intravesical chemotherapy. Specifically, he receives a single instillation of chemotherapy into the bladder. Cylindrical particles as described herein could include mitomycin C (MMC), gemcitabine, docetaxel, carboplatin and cisplatin as an active agent. The particles are administered in a stacked arrangement using a catheter. After insertion, the particles revert to a retention configuration. The particles remain in the bladder as the MMC is released into the bladder and nearby tissue.

After about ten days, the particles disassemble as the particles separate from one another. The individual components are expelled in the patient's urine. The provider conducts a second visual inspection of the bladder via cystoscopy. The number and size of the tumors has decreased substantially (i.e., by about 85%). The provider suggests immunotherapy and regular monitoring of the patient's bladder.

### Example 7

### Intravesical Particles to Treat Non-Muscle Invasive Bladder Cancer (NMIBC) (4RC Construct)

In this example, an 82-year-old male patient visits a healthcare provider and complains of blood in the urine, pain with urination, and low back pain. BCG-unresponsive disease refers to high-grade non-muscle invasive bladder cancer (NMIBC) that persists or recurs despite adequate Bacillus Calmette-Guérin (BCG) treatment. The provider conducts a visual inspection of the bladder via cystoscopy. Pathological examination confirms the presence of tumor cells and the patient is diagnosed with non-muscle invasive bladder cancer (NMIBC).

The patient receives intravesical chemotherapy. Specifically, he receives a single instillation of chemotherapy into the bladder. Cylindrical particles as described herein could include gemcitabine, docetaxel, carboplatin and cisplatin as an active agent. The particles are administered in a stacked arrangement using a catheter. After insertion, the particles revert to a retention configuration. The particles remain in the bladder as the agent is released into the bladder and nearby tissue.

In this example, the particles are configured to administer a “burst” of agent for an induction phase of 7 to 10 days. This is followed by a lower amount of the agent released during a maintenance phase. The particles release gemcitabine during at a relatively lower concentration for a longer duration (e.g. 30 days).

After about 45 days, the particles disassemble as the particles separate from one another. The particles are expelled in the patient's urine. The provider conducts a visual inspection of the bladder via cystoscopy. The number and size of the tumors has decreased substantially (i.e., by about 85%). The provider suggests immunotherapy and regular monitoring of the patient's bladder.

A heterogeneous configuration in which each cylinder has the same polymer composition. Here, one cylinder contains a different active agent than the other three cylinders. In this example, gemcitabine is delivered in three cylinders and docetaxel is delivered in the other cylinder, as these chemotherapeutic agents are synergistic in the treatment of bladder cancer.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

What is claimed is:

1. An intravesical drug delivery device, comprising:
a) a plurality of elongate modules, each module comprising a polymeric matrix and at least one active agent disposed within the polymeric matrix, within a coating on the polymeric matrix, or both;
b) one or more hinge elements coupling adjacent modules to form a chain structure;
wherein the hinge elements are configured to: (a) permit bending of the chain in a first direction to enable transurethral insertion; (b) resist bending in a second direction to inhibit expulsion during voiding; and (c) undergo controlled weakening or degradation in a fluid environment;
wherein the device is configured to assume:
(i) an insertion configuration, in which the modules are arranged substantially collinearly, having a maximum cross-sectional profile of less than about 10 mm for transurethral administration;
(ii) a retention configuration, in which at least two modules are oriented non-collinearly with respect to one another by the hinge elements, having a minimum cross-sectional profile of at least about 16 mm in any orientation so as to resist excretion through the urethra; and
(iii) an elimination configuration, after detachment of one or more modules, in which each individual module has a maximum cross-sectional profile of less than about 6 mm for passive excretion through the urethra; and
wherein the hinge elements are further configured to permit detachment of the modules, wherein the detachment may be sequential, simultaneous, or a combination thereof.

2. The intravesical drug delivery device of claim 1, wherein each module is rod-shaped.

3. The intravesical drug delivery device of claim 2, wherein each rod-shaped module has a diameter of about 2 mm to about 5 mm.

4. The intravesical drug delivery device of claim 2, wherein each rod-shaped module has a length of about 5 mm to about 30 mm.

5. The intravesical drug delivery device of claim 1, wherein the polymeric matrix comprises glyceryl tripalmitate (GTP), polycaprolactone (PCL), poly(caprolactone-co-glycolide) (PCL-GA), poly(lactide-co-glycolide) (PLGA), ethylene-vinyl acetate (EVA), or combinations thereof.

6. The intravesical drug delivery device of claim 1, wherein detachment of the modules occurs upon exposure to liquid after a period of 12 hours to 90 days.

7. The intravesical drug delivery device of claim 1, wherein each of the one or more hinge elements comprises a biodegradable polymer.

8. The intravesical drug delivery device of claim 7, wherein the biodegradable polymer comprises poly(caprolactone-co-glycolide) (PCL-GA).

9. The intravesical drug delivery device of claim 8, wherein the PCL-GA has a ratio of caprolactone to glycolide from about 10:90 to about 90:10.

10. The intravesical drug delivery device of claim 1, wherein each of the one or more hinge elements includes an asymmetric geometry that permits bending in the first direction and resists bending in the second direction.

11. The intravesical drug delivery device of claim 1, wherein each module has a density between about 0.80 g/cm³ and less than about 1.05 g/cm³.

12. The intravesical drug delivery device of claim 1, wherein the plurality of elongate modules is comprised of a first module and a second module,
wherein the first module is configured for a burst-release of the active agent and the second module is configured for sustained-release of the active agent.

13. The intravesical drug delivery device of claim 1, wherein release kinetics of the at least one active agent are controlled by one or more of: (a) module position within the chain; (b) polymer composition; (c) coating thickness; or (d) hinge degradation timing.

14. The intravesical drug delivery device of claim 1, wherein different modules within the device are coated with at least one polymer selected from EVA, PCL-GA, polyurethane, PLGA, or blends thereof,
wherein one or more of the coatings further comprises at least one active agent such that the active agent is released as the coating degrades, and
wherein the different modules possess different coating thicknesses, porosities, or compositions, thereby enabling one or more of: (a) burst release from a first module, (b) sustained release from a second module, (c) delayed or pulsatile release from a third module, or (d) multi-drug sequential release.

15. The intravesical drug delivery device of claim 1, wherein the at least one active agent is selected from an anti-infective agent, an anesthetic agent, an analgesic agent, a diuretic, an anti-inflammatory agent, a coagulant or an anti-coagulant, a chemotherapeutic agent, an agent for the treatment of incontinence, a renin-angiotensin-aldosterone system (RAAS) inhibitor, an immunomodulating agent, an agent for treating kidney stones, an agent for gene therapy, or a contrast agent for diagnostics and monitoring.

16. The intravesical drug delivery device of claim 1, wherein one or both ends of at least one module include a concave feature configured to increase joint contact area with an adjacent module or hinge element, improve bond strength, and promote more predictable joint degradation behavior.

17. The intravesical drug delivery device of claim 1, comprising two or more limbs, each limb comprising two or more modules connected end-to-end by biodegradable in-line joints, wherein the limbs are connected to one another by one or more angled hinge joints, and wherein the in-line joints and the angled hinge joints have different degradation rates so as to enable sequential or staged detachment of individual modules.

18. The intravesical drug delivery device of claim 1, wherein each module is encased in a polymeric casing applied by one or more of dip-coating, spray-coating, wrapping a pre-formed polymer sheet or film, co-extrusion, or placement into a pre-formed polymer sleeve, wherein the casing serves at least two of:
a) controlling the rate and duration of active agent release from the module,
b) protecting the module against mechanical breakage during retention in the bladder, lengthening the release profile of the active agent relative to an uncoated module, and
c) serving as a drug-delivery matrix comprising at least one active agent that is released as the casing degrades,
wherein in such configurations the rod core may be substantially free of active agent and serve primarily as a structural or buoyancy element.

19. The intravesical drug delivery device of claim 1, wherein the device comprises four rod-shaped modules arranged as two limbs of two modules each, each limb connected end-to-end by a biodegradable in-line joint, and the two limbs connected by an angled hinge joint,
wherein the in-line joints and the angled hinge joint comprise poly(caprolactone-co-glycolide) (PCL-GA) formed by controlled deposition, and wherein each rod-shaped module is encased in a polymeric casing that is distinct from the joint and hinge material, the casing comprising one or more of EVA, PCL-GA, PLGA, or PCL, and optionally further comprising at least one active agent.

20. A method of manufacturing a modular intravesical drug-delivery device, the method comprising steps of:
a) forming a polymer-drug composition into elongate modules by one or more of hot-melt extrusion, injection molding, compression molding, direct compression, 3D printing, or solvent casting;
b) cutting the elongate modules into segments of variable length;
c) applying a casing or coating to the segments via one or more of dip-coating, spray-coating, wrapping a pre-formed polymer sheet or film, co-extrusion, placement into a pre-formed polymer sleeve, or solvent-based coating, wherein the casing or coating optionally comprises at least one active agent;
d) coupling adjacent modules with one or more hinge elements using heat-setting, solvent welding, or mechanical fixation to form a chain structure; and
e) configuring the hinge elements to permit directional bending and to degrade or weaken in a fluid environment to enable staged detachment of the modules.

21. The method of claim 20, wherein the casing or coating comprises one or more of polycaprolactone (PCL), poly(caprolactone-co-glycolide) (PCL-GA), polycaprolactone-lactide (PCL-LA), or PEG-PCL block copolymers applied in one or more layers to create a flexible and biodegradable protective barrier.

22. The method of claim 20, further comprising a quality control step of verifying drug uniformity, coating integrity, hinge integrity, and mechanical break strength of the coated modules before assembly into the chain structure.

23. The method of claim 20, wherein the method produces a four-module two-limb intravesical device by: (a) forming rod-shaped modules by compression molding of a polymer composition; (b) applying a polymeric casing to each module by dip-coating one or more layers of one or more of EVA, PCL-GA, PLGA, or PCL, the casing optionally comprising at least one active agent; and (c) forming in-line joints within each limb and an angled hinge joint between the two limbs by controlled deposition of poly(caprolactone-co-glycolide) (PCL-GA).

24. A method of treating and/or preventing a disorder of the urinary system in a subject, the method comprising:
a) administering an intravesical drug delivery device into a bladder of the subject via transurethral delivery, the device comprising a plurality of elongate modules each comprising a polymeric matrix and at least one active agent disposed within the polymeric matrix, within a coating on the polymeric matrix, or both, coupled by one or more hinge elements to form a chain structure;
b) retaining the device within the bladder in a retention configuration in which at least two modules are oriented non-collinearly so as to resist excretion through the urethra;
c) releasing the active agent from the modules during a retention period; and
d) permitting detachment of the modules by controlled weakening or degradation of the hinge elements, wherein the detached modules are dimensioned for passive excretion through the urethra during urination.

25. The method of claim 24, wherein the disorder is a urinary tract infection, bladder cancer, kidney cancer, ureter cancer, urethra cancer, overactive bladder, underactive bladder, retained urine, kidney failure, interstitial cystitis, or stress urinary incontinence.

26. The method of claim 24, wherein the retention period is more than one month.

27. The method of claim 24, wherein the device is administered through a catheter or vesical sheath in the insertion configuration having a maximum cross-sectional profile of less than about 10 mm.

28. The method of claim 24, wherein the detachment of the modules is sequential, with different hinge elements degrading at different rates so as to provide staged elimination of the modules.

29. The method of claim 24, wherein administering the device comprises inserting a four-module two-limb chain device through a catheter, the device comprising two pairs of rod-shaped modules connected within each pair by biodegradable in-line joints of poly(caprolactone-co-glycolide) (PCL-GA) and between the pairs by an angled hinge joint of PCL-GA, wherein during the retention period the in-line joints degrade before the angled hinge joint so as to release individual rod-shaped modules sequentially for passive excretion through the urethra.

30. The method of claim 24, wherein the active agent is disposed within the coating on the polymeric matrix and the polymeric matrix of each module is substantially free of active agent, such that the module core serves primarily as a structural or buoyancy element and the active agent is released as the coating degrades during the retention period.
